(19) United States
(12) Patent Application Publication
(10) Pub. No.: US 2026/0028669 A1
(43) Pub. Date: Jan. 29, 2026

(54) METHODS AND COMPOSITIONS FOR NUCLEIC ACID ANALYSIS

(71) Applicant: CHAPTER DIAGNOSTICS, INC., Menlo Park, CA (US)

(72) Inventors: Chunlin Wang, Menlo Park, CA (US); Zhihai Ma, Mountain View, CA (US); Baback Gharizadeh, Menlo Park, CA (US)

(73) Assignee: CHAPTER DIAGNOSTICS, INC., Menlo Park, CA (US)

(21) Appl. No.: 19/099,917

(22) PCT Filed: Jul. 28, 2023
(86) PCT No.: PCT/US2023/028985

§ 371 Date: Jan. 30, 2025

Related U.S. Application Data
(60) Provisional application No. 63/369,946, filed on Jul. 30, 2022.

(51) Int. Cl. C12Q 1/6869; C12Q 1/6813; C12Q 1/686; C12Q 1/6886; C12Q 1/689; C12Q 1/6895; C12Q 1/70

(52) U.S. Cl. CPC C12Q 1/6869; C12Q 1/6813; C12Q 1/686; C12Q 1/6886; C12Q 1/689; C12Q 1/6895; C12Q 1/701; C12Q 2600/16; C12Q 2600/166

(57) ABSTRACT

The present disclosure relates to compositions and methods for detection, identification, sequence analysis and quantification of biological organisms in a single amplification reaction. The disclosed method utilizes next-generation sequencing (NGS) to sequence amplified products. The present disclosure is also directed to kits containing primers specific to microbial and viral organisms, cancer, genetic disorders and forensics.

### RELATED APPLICATIONS

This application claims priority to U.S. Provisional Patent Application No. 63/369,946 filed on Jul. 30, 2022, titled “Multiplex Barcoding Amplification and Multilocus Sequence Typing”, the entire content of which is incorporated herein.

### BACKGROUND OF THE INVENTION

The multiplex PCR (polymerase chain reaction) method allows for the simultaneous amplification of multiple target DNA sequences in a single reaction. It involves the use of multiple primer pairs, each specific to a different target sequence, along with a DNA polymerase enzyme and nucleotides. By incorporating multiple primer sets, each corresponding to a specific target region, into a single PCR reaction, multiple DNA fragments can be amplified simultaneously.

Multiplex PCR has broad applications in various fields, including medical diagnostics, genetics, forensics, microbial and viral detection, and research. Some applications of multiplex PCR include: (1) genetic disease screening; (2) pathogen identification; (3) forensic DNA analysis; (4) human leukocyte antigen (HLA) typing; and (5) cancer mutation analysis.

For genetic disease screening, multiplex PCR can be used to screen for the presence of mutations or genetic variations associated with specific inherited diseases, genetic disorders, or cancer. By simultaneously amplifying multiple target genes or regions, it enables the detection of multiple disease-associated variants in a single test. For pathogen identification, multiplex PCR can be employed for the rapid detection and identification of pathogens, such as bacteria, viruses, parasites, or fungi, in clinical, food or environmental samples. By targeting specific genomic regions unique to different pathogens, multiplex PCR can provide a rapid and accurate diagnosis. For forensic DNA analysis, multiplex PCR is widely used in forensic science for DNA profiling and identification, enabling the amplification of multiple genetic markers, such as short tandem repeats (STRs), thereby allowing for the simultaneous analysis of multiple loci and enhancing the accuracy of DNA profiling. For HLA typing, which is crucial in organ transplantation and immunogenetics, multiplex PCR-based methods can simultaneously amplify and identify specific HLA gene alleles, enabling rapid and comprehensive HLA typing for compatibility assessment. For cancer mutation analysis, multiplex PCR can be employed to detect specific mutations or genetic alterations associated with cancer. By amplifying target genes known to harbor cancer-associated mutations, multiplex PCR allows for efficient screening and profiling of tumor samples.

Targeted DNA sequencing allows the user to selectively analyze specific regions of a genome. Instead of sequencing the entire genome, only specific regions of interest are sequenced. This approach is more focused and efficient, as it allows people skilled in the art to gather information about specific genes or genomic regions without sequencing the entire genome. Targeted DNA sequencing typically involves the use of capture probes or primers that are designed to specifically bind to and capture the regions of interest. These primers or probes are often complementary to the DNA sequences being targeted, enabling their selective amplification and sequencing. This is particularly useful when studying specific genes or genomic regions that are known to be associated with certain pathogenicity, diseases, or traits. By targeting these regions, researchers can analyze variations, mutations, or structural changes that may be relevant to a particular condition or characteristic. Compared to whole genome sequencing (WGS), targeted DNA sequencing is faster, less expensive, and requires less computational resources. It has become a valuable tool in various research areas, including cancer genomics, genetic disease diagnosis, forensics, and personalized medicine.

Detection as well as accurate identification to subtype level of pathogens involve various methods and techniques. The ability to distinguish accurately between different strains and sub-strains within bacterial/fungal/parasite/viral species is an important requirement for accurate genotyping and epidemiological surveillance especially in agricultural and food industries and in clinical settings. Globalization has increased the complexity of the food supply chain as more than ever; companies are relying on ingredient and raw materials suppliers from more regions of the world. This in turn has led to a potential increase in risk associated with the food as the raw materials may not be produced under the same hygienic standards as those produced domestically. This has resulted in changes in food safety regulations, for both domestic and foreign food suppliers, requiring them to identify all potential hazards associated with the food and implement measures to prevent these risks from becoming a risk to the public health. Despite these measures, food safety recalls due to product contamination as well as outbreaks, especially those concerning pathogenic microorganisms, continue to occur at significant cost to the public and food manufacturers. Among the preventive measures identified is the need for better technologies that can allow various players within the food supply chain to proactively detect, identify, and remove contaminated foods.

Although selective housekeeping genes are utilized for strain differentiation, slow accumulation of variation within housekeeping loci will lead to a lack of discrimination between very closely related strains. Therefore, more diverse loci with informative variations can provide high-resolution data that can results in genotyping to sub-strain level. Currently, the food industry needs a straightforward, efficient, widely applicable, and cost-efficient method to not only detect, but also determine serovar and potential sources of multiple strains in food and environment. For instance, Salmonella genus includes more than 2,700 serotypes, which cannot be differentiated with low-resolution methods. While whole genome sequencing (WGS) can generate complete genomic profile of food pathogens, it is a laborious, time-consuming, and expensive method that necessitates pure isolates. As a result, WGS is not applicable to samples with complex microbial background, limiting its widespread application by food industry.

Bacterial, fungal, parasite, or viral subtyping determines the similarity between separate isolates of the same species, which is an important measure in preventing foodborne illness outbreaks, as it allows public health officials to quickly identify the source of contamination and take appropriate action to prevent further spread. There are several methods of microbial and viral subtyping that are currently being used. Serotyping is a phenotypic typing method that involves identifying the type of antigens on the surface of the bacterial cell. This method can be used to differentiate between different strains of a species, but it is less discriminatory than molecular methods. Pulsed-field gel electrophoresis (PFGE) is a molecular typing method that uses restriction enzymes to digest bacterial DNA into large fragments, which are then separated by size using gel electrophoresis. The resulting banding patterns can be used to compare bacterial strains and identify relatedness.

Multilocus Sequence Typing (MLST) is a molecular typing method that involves sequencing several housekeeping genes within a microbial and viral genome. The resulting sequences can be compared with the sequences from other relevant strains/types to identify their relatedness and to infer their evolutionary relationships. Traditional MLST classifies and compares bacterial strains based on the nucleotide sequences of several gene loci. The method involves PCR amplification and sequencing internal fragments of 6-8 housekeeping genes, which are genes that encode for essential metabolic functions and are conserved among bacterial species. Although traditional MLST can differentiate between bacterial strains based on sequence variations at targeted loci, it may not be able to resolve closely related strains. While traditional MLST has several advantages, it has some limitations such as: (1) limited genomic coverage, given that MLST only targets a small number of genes, usually six to nine, which may not provide a comprehensive view of the genetic diversity of the bacteria/fungi/parasites/viruses; (2) insufficient discriminatory power, given that MLST may not always be able to distinguish closely related strains, which can be problematic in epidemiological studies or outbreak investigations; and 3) MLST requires a pure colony, and samples with complex background cannot be analyzed.

WGS is a powerful technique for studying the genetic makeup of an organism, including microbial and viral genomes. This method provides the highest level of resolution and can be used to identify SNPs and other genetic variations within the pathogen. WGS can provide the highest level of specificity and sensitivity in identifying microbial and viral subtypes and tracking the spread of outbreaks. However, there are some limitations to this technique. First, the cost of WGS can be a limiting factor, especially for large-scale studies or routine clinical applications. However, the cost of sequencing has been decreasing rapidly in recent years, making it more accessible to researchers and clinicians but still expensive for routine settings. Second, the amount of data generated by WGS can be massive, which can pose challenges for storage, processing, and analysis. Handling and analyzing such large datasets require specialized computational resources and expertise. Third, the quality of the sequencing reads can affect the accuracy of the results, and there may be technical issues or errors during the sequencing process that need to be addressed. Fourth, interpreting the results of WGS can be challenging, especially for non-experts and routine laboratories, as it requires a detailed understanding of genomics, bioinformatics, and statistical analysis. There is also a risk of misinterpretation or incorrect conclusions due to the complexity of the data. Fifth, WGS is not always able to resolve fine-scale variation within microbial populations, such as single nucleotide variants or small indels, which can lead to difficulties in accurately tracking transmission or understanding the evolution of bacterial populations. Sixth, WGS requires pure colony for microbial and viral genome sequencing. Samples with complex background cannot be analyzed by WGS. In brief, while WGS is a powerful and increasingly accessible tool for microbial, fungal, parasitic, and viral genomics, it requires careful consideration of its limitations and potential challenges in data management, analysis, and interpretation, which in return demands for skillful data analysis expertise and workforce.

Instead of sequencing low discriminatory housekeeping genes by traditional MLST, a solution is to simply sequence a large panel of informative variable/hypervariable regions across a microbial and viral genome for significantly higher discriminatory resolution. The number of these polymorphic loci differ on the bacterial, fungal, parasitic, and viral strains analyzed. In general, up to 100 loci may suffice for discrimination to sub-strain level, however, the invention presented herein is not limited to the number of loci or regions. The loci number could be 20, 50, 100, 200, or more. The sequence data from such variable data can provide differentiative information about species, strain, sub-strain, virulence potential in human infection, monitoring of drug resistance, and tracking the source of infection from human, animal, food, and environmental reservoirs without the need for WGS.

Timely and accurate screening, detection, typing and subtyping minimizes the health and economic burden of food, animal and environmental pathogens. There remains a need for effective and cost-efficient high-resolution methods. In addition, such methods should involve a simple workflow and be easily automated in order to bypass WGS complex workflow and hurdles.

### SUMMARY OF THE INVENTION

In some embodiments, the present disclosure describes a method of screening and analyzing at least one sample for food, animal, human and environmental pathogens, comprising the steps of: for each sample, hybridizing a plurality of target-specific primers with nucleic acid from the sample in the presence of barcoded universal primers to form a test reaction in a single reaction container, wherein at least one target-specific primer is configured to bind to at least one target nucleic acid sequence; subjecting each test reaction to amplification conditions to generate amplicons; pooling amplicons from each sample; subjecting at least a portion of the pooled amplicons to bead cleanup to form enriched amplicons; and sequencing the pooled and enriched amplicons, formed from each sample, by next-generation sequencing.

In some embodiments, each barcoded universal primer comprises: a universal priming portion at the 3′-end; a barcode portion in the middle; and a universal priming portion at the 5′-end. In some embodiments, each target-specific primer comprises a specific sequence portion directed to a target nucleic acid sequence and a universal priming portion. In some embodiments, each sample is obtained from a subject, food, one or more plants, or an environmental source. In some embodiments, the target-specific primers comprise primers configured to amplify at least 20 variable regions of at least a bacterium, fungi, parasite or virus for identification, genotyping, subtyping and detection of co-presence of multiple isolates.

In some embodiments, the method described herein further comprises mapping-and-counting for microbial and viral typing, subtyping, and surveillance of multiple pathogen genomes, comprising the additional steps of: (1) determining the score for a locus A for a first genome as the ratio between the number of unique reads mapped onto the first genome's locus A and the total number of unique reads mapped onto the locus A for the first genome and at least one other genome wherein if no read is mapped to the first genome's locus A, presetting the score to a de minimis number; repeating the determination step of part (1) for at least one other locus; and (3) determining an overall score for the first genome by multiplying the scores for all tested loci from steps (1) and (2). In some embodiments, the method further comprises the steps of: (a) determining the genomes with the highest overall score on any remaining reads; (b) ending the assessment if the number of non-empty loci for highest-scored genome is less than a preset cutoff; (c) outputting the highest-scored genomes; (d) removing reads mapped to all then-highest scored genomes; and (e) repeating steps (a)-(d) until the assessment ends in accordance with step (b). In some embodiments, the preset cutoff is at least 10.

In some embodiments, the target-specific primers comprise primers configured to amplify multiple variable regions of at least a bacteria, fungi, parasite or virus for genotyping, subtyping, detection and identification of multiple genotypes, serotypes or subtypes of the same species or different species in the same sample. In some embodiments, the target-specific primers comprise primers configured to amplify and detect target sequences of at least one species, type or subtype of bacteria, fungi, parasites, or viruses in the same sample.

In some embodiments, the sample comprises at least one microbial and/or viral species, strain or sub-strain.

In some embodiments, the target-specific primers comprise primers configured to amplify and analyze target sequences related to forensic testing.

In some embodiments, the method further comprises the step of pooling the enriched amplicons from each sample prior to sequencing. In some embodiments, the method further comprises the step of quantifying each type and species in each sample after sequencing the enriched amplicons.

In some embodiments, the test reaction comprises a polymorphic gene with unique sequence for an internal control.

In some embodiments, the present disclosure describes a method of screening at least one sample for food, animal, human, plant, and environmental pathogens, comprising the steps of: for each sample, hybridizing a plurality of target-specific primers with nucleic acid from the sample in the presence of barcoded universal primers to form a test reaction in a single reaction container, wherein at least one target-specific primer is configured to bind to at least one target sequence selected from the group consisting of: bacteria, fungi, parasites or virus, wherein each barcoded universal primer comprises: a universal priming portion at the 3′-end; a barcode portion in the middle; and a universal priming portion at the 5′-end; and wherein each target-specific primer comprises a specific sequence portion directed to a target nucleic acid sequence and a universal priming portion; subjecting each test reaction to amplification conditions to generate amplicons; pooling amplicons from each sample; subjecting at least a portion of the pooled amplicons generated from each sample to bead cleanup to form enriched amplicons; and sequencing the pooled and enriched amplicons, formed from each sample, by next-generation sequencing.

In some embodiments, the present disclosure describes a method of screening at least one sample for forensic DNA analysis, cancer, or genetic disorders, comprising the steps of: for each sample, hybridizing a plurality of target-specific primers with nucleic acid from the sample in the presence of barcoded universal primers to form a test reaction in a single reaction container, wherein at least one target-specific primer is configured to bind to at least one target sequence, wherein each barcoded universal primer comprises: a universal priming portion at the 3′-end; a barcode portion in the middle; and a universal priming portion at the 5′-end; and wherein each target-specific primer comprises a specific sequence portion directed to a target nucleic acid sequence and a universal priming portion; subjecting each test reaction to amplification conditions to generate amplicons; pooling amplicons from each sample; subjecting at least a portion of the pooled amplicons generated from each sample to bead cleanup to form enriched amplicons; and sequencing the pooled and enriched amplicons, formed from each sample, by next-generation sequencing.

In some embodiments, the present disclosure describes a kit, comprising multiplex target-specific primers configured to bind to target sequences specific to: biological samples related to cancer, genetic disorders, forensic testing, and microbial and viral species.

In some embodiments, the present disclosure describes methods and compositions of amplifying selective target region(s) in a nucleic acid sample. In some embodiments, the method comprises the steps of: (1) contacting the nucleic acid sample with target-specific primers in PCR reaction, in presence of barcoded universal primers; and (2) allowing primer extension to generate target amplification products (amplicons) of different sizes. In some embodiments, the method comprises the step of determining the presence or absence of target amplification product. In some embodiments, the method comprises the step of establishing the sequence of the target amplification products. In some embodiments, less than 50, 40, 30, 20, 10, 5, 0.5, or 0.1% of the amplified products are primer-dimers or artifacts.

In some embodiments, the concentration of each target-specific primer can be about 500, 250, 100, 80, 70, 50, 30, 10, 2, or 1 nM. In some embodiments, the GC content of the target-specific primers can differ, and as an example it can be between 40% and 70%, or between 30% and 60% or 50% and 80%. In some embodiments, the melting temperature (Tm) of the target-specific primers can be between 55° C. and 65° C., or 40° C. and 70° C., or 55° C. and 68° C. In some embodiments, the length of the target-specific primers can be between 20 and 90 bases, 40 and 70 bases, 20 and 40 bases or 25 and 50 bases. In some embodiments, the 5′-region of the target-specific primer is a universal primer binding site that is not complementary or specific for any nucleic acid region in the sample. In some embodiments, the length of the target amplicons is between 50 and 500 bases, 90 and 350 bases, or 200 and 450 bases.

In various embodiments of any of the aspects of the present disclosure, the method of primer extension is based on the state-of-art polymerase chain reaction (PCR). In various embodiments, annealing time can be greater than 0.5, 1, 2, 5, 8, 10 or 15 minutes. In various embodiments, extension time can be greater than 0.5, 1, 2, 5, 8, 10 or 15 minutes.

In some embodiments, the method disclosed herein quantifies the copy number of the target sequence present in the sample.

In various embodiments of the present disclosure, the compatibility and non-compatibility score of the selected primers are calculated based on different factors of target amplicon GC content, target amplicon melting temperature, target amplicon heterozygosity rate, complementary rate of the candidate primer for the target region; candidate primer size, target amplicon size and amplification efficiency and off-target rate. The selected target-specific primers can hybridize to the nucleic acid target and selectively amplify the target regions. In various embodiments, the test sample is from a subject, individual, food, plant, animal, soil or environment that is suspected to have an infection or disease, or an increased risk for an infection or disease; and wherein one or more of the target nucleic acid comprise a sequence at the target region associated with an infection or disease or increased risk of an infection or disease. In some embodiments, the test sample is from a subject, individual, animal, soil or environment not related to any diseases or infections. The profile of target regions can serve as identity mark for a subject, individual, animal, or other sample, in a way similar to fingerprint. In some embodiments, information can be used for disease screening, detection, disease management, pathogen surveillance, food recalls, outbreaks or pandemics.

In one embodiment, the method disclosed herein can be used to screen, detect and identify microbial and viral strains and types. In one embodiment, the method disclosed herein can be used to screen, detect, genotype, serotype, subtype and trace the source of infection (surveillance) using an extensive MLST approach targeting variable/hypervariable regions. In some embodiments, the candidate primers contact the nucleic acid sample; wherein the forward strand and reverse strand target-specific primers hybridize to target regions (if present in the sample), where the nucleic acid sample may have microbial and/or viral organisms or is suspected to have microbial and/or viral organisms, amplifying a plurality of target nucleic acids; subjecting the amplicons to next-generation sequencing; and analyzing the sequence data by software analysis. In some embodiments, the detected infections can be clinically actionable. In some embodiments, detected infections can be associated with drug resistance. In some embodiments, detection, identification, and quantitation of microbial and viral species, strains, and sub-strains can be related with disease. In some embodiments, the biological sample can be monitored for source of infection or surveillance.

In one aspect, the method and composition disclosed herein is designed to detect, identify, and quantify target nucleic acids in a sample that may contain microbial and viral organisms. In some embodiments the disclosed method comprises the steps of: (1) contacting the nucleic acid targets in a sample with primers, wherein forward strand and reverse strand target-specific primers hybridize to different target regions in the test reaction; (2) amplifying the target nucleic acids under optimal amplification conditions; (3) sequencing the amplified products by NGS; and (4) analyzing and quantitatively measuring the generated sequence reads by a mapping-and-counting methodology

In one embodiment, the method disclosed herein can be used to screen and analyze target regions of a genome for disease such as cancer or genetic disorder. In one embodiment, the method disclosed herein can be used for analyzing a genome for forensic DNA analysis based on the DNA profile such as short tandem repeat (STR) regions. In some embodiments, the method disclosed herein can be used for pharmacogenetics or drug resistance to detect the genetic variations that influence an individual's response to medication. In some embodiments, the candidate primers contact the nucleic acid sample; wherein the forward strand and reverse strand target-specific primers hybridize to target regions, amplifying a plurality of target nucleic acids; subjecting the amplicons to next-generation sequencing; and analyzing the sequence data by software analysis. In some embodiments, the detected nucleic acid variations can be clinically actionable. In some embodiments, the biological sample can be monitored for prognosis.

In some embodiments, the nucleic acid sample comprises genomic nucleic acid. In some embodiments, the sample comprises nucleic acid molecules obtained from food, vegetables, produce, plants, soil, spoilage, water, environment, or food production facilities. In some embodiments, the sample comprises nucleic acid molecules obtained from urine, tissue, saliva, biopsies, sputum, swabs, surgical resections, cervical swabs, tumor tissue, fine needle aspiration (FNA), scrapings, swabs, mucus, semen, other non-restricting clinical or laboratory obtained samples.

In another aspect, the present disclosure is directed to kits comprising target-specific primers for amplifying target regions of interest in a sample.

In some embodiments, the disclosed method comprises the steps of: performing one-step multiplex barcoding amplification, and sequencing the resulting amplicons by NGS. In certain embodiments, the samples are obtained from subjects with single or multiple co-infections. In some embodiments, the method's analytical sensitivity is 10 copies for each microbial and viral species in a sample; the highly multiplex PCR amplifies 20, 50, 100, 200, 500, 1,000 or more targets with minimal primer-primer interactions. In some embodiments, the method comprises the step of performing single-reaction, single-step barcoding multiplex PCR. In some embodiments, the method can analyze 5, 10, 20, 50, 100, 200, 500, 1,000, 2,000, 5,000, 10,000 or more samples by a single NGS sequencing run.

In some embodiments, the disclosed method comprises the use of internal control(s) for each sample in the reaction test, wherein the internal control is often a housekeeping gene, a mitochondrial sequence or a target sequence and monitors amplification and acts as normalization, qualification or quantification control.

In some embodiments, the disclosure relates to methods, compositions, and kits for application of multiplex target amplification and target enrichment prior to downstream analysis such as next generation sequencing. The method relies on using a plurality of target-specific primers and target enrichment amplification in a DNA sample that is suspected to have disease (cancer or genetic disorder), drug resistance, forensics information, or microbial and viral pathogens. The target-specific primers amplify the target nucleic acids under optimal conditions in presence of amplification reagents such as polymerase and dNTPs to at least amplify one or more nucleic acid targets of interest.

In some embodiments, the disclosure relates to a composition comprising a plurality of target-specific primers that contact the target sequences in the sample and have complementary sequences to target DNA related (non-limiting) to disease (cancer/genetic disorders), drug resistance, forensics or microbial and viral organisms.

In some embodiments of the disclosed method, the primer design methodology selects the candidate target-specific primers based on steps of: (1) extracting genomic sequences of a microbial or viral organisms; (2) designing a set of target-specific forward strand and reverse strand target-specific primers for variable, hypervariable, housekeeping or other target sequences with proper GC content, Tm, and varying distances from each targeted region; (3) for each primer, searching target genome sequences for off-target matches; filter primers and keep those primers that pass the off-target threshold; (4) searching the 3′-end portion of each primer for complementary matches with primer sequences of the set; filter primers progressively where the primer with its 3′-end having most complementary matches is removed first; and (5) synthesizing primers and running the entire wet-lab experiment using next-generation sequencing; calibrate the performance of each primer and filter out primers of undesired performance. In some embodiments, the primer selection procedure steps 2 to 4 and steps 2 to 5 are repeated until each target sequence is covered by at least one forward strand target-specific primer and one reverse strand target-specific primer in the primer set.

In various embodiments of any of the aspects of this disclosure, the methods and compositions feature multiplex amplification and target enrichment of target nucleic acid regions. In some embodiments, the disclosed method comprises the steps of: (1) contacting target-specific primers with target nucleic acid sequences in presence of barcoded universal primers and hybridizing to target nucleic acid sequences in the sample; (2) subjecting the test reaction to amplification under optimal amplification conditions; (3) pooling together the amplified products from each individual sample; (4) subjecting a portion of the pooled amplified products to bead cleanup to remove unconsumed primers and primer-dimers and create enriched amplified products; (5) subjecting a portion of enriched amplified products to standard normalization and quantification; and (6) sequencing the amplicon by next-generation sequencing.

In one embodiment, the barcoded universal primers comprise: a) a universal priming portion at the 3′-end; b) a barcode portion in the middle; and c) a universal priming portion at the 5′-end (FIG. 1). In one embodiment, each target-specific primer comprises a specific sequence portion directed to target nucleic acid sequence and a universal priming portion.

In some embodiments, the composition comprises a plurality of target-specific primers wherein at least one target-specific primer is at least 90% identical to any one of the nucleic acid targets. In some embodiments, the composition comprises a plurality of target-specific primers having a sequence identity of at least 91%, 92%, 93%, 94%, 95%, 96%, 97%, 98% or 99% to the nucleic acid targets in the sample.

In some embodiments, the disclosure relates to a composition comprising a plurality of target-specific primers wherein the sequence complementary to target nucleic acid of interest is about 15 to 40 bases in length.

In some embodiments, the disclosure relates to a composition of pre-calculated design of target primers that generate minimal cross-hybridization or primer-primer interactions with other target specific primers in the composition. In some embodiments, the primers in the composition are designed to avoid non-specific priming that can lead to non-specific amplifications. In some embodiments, the amplification conditions such as annealing temperature, annealing duration and primer concentrations can be adjusted to minimize amplification artifacts such as primer-dimers.

In some embodiments, the disclosure relates to a method or composition comprising a plurality of target-specific primers having minimal cross-hybridization to non-specific sequences present in the sample. In some embodiments, such cross-hybridization to non-specific targets could be monitored and evaluated by downstream analysis such as next generation sequencing.

In some embodiments, the disclosure relates to a method or composition comprising a plurality of target primers having minimal self-complementary structure. In some embodiments, the composition comprises at least one target-specific primer that do not form a secondary structure, such as hairpins or loops. In some embodiments, the composition comprises a plurality of target-specific primers that the majority, or potentially all the target-specific primers do not form secondary structures such as hairpins and loops.

In some embodiments, the target nucleic acid is obtained from food, fresh produce, water, soil, spoilage, environment, or a biological sample from a subject. In some embodiments, the sample comprises proteins, cells, fluids, biological fluids, preservatives, and/or other substances. In certain embodiments, the sample is originates from urine, tissue, saliva, biopsies, sputum, swabs, surgical resections, cervical swabs, tumor tissue, fine needle aspiration (FNA), scrapings, swabs, mucus, semen, other non-restricting clinical or laboratory obtained samples.

In some embodiments, the target amplification products are sequenced by next generation sequencing on current state-of-art next-generation sequencing technologies or platforms such as Illumina platforms (reversible dye-terminator sequencing), 454 pyrosequencing, Ion Semiconductor sequencing (Ion Torrent), PacBio SMRT sequencing, Qiagen GeneReader sequencing technology, Oxford Nanopore sequencing and Element Biosciences sequencing technology. In some embodiments, the disclosed method is not limited to these next-generation sequencing technologies examples and can be applied to new sequencing innovations.

In certain embodiments, the foregoing methods may be performed at multiple time points.

### BRIEF DESCRIPTION OF THE DRAWINGS

The disclosure can be better understood with reference to the following drawings. The elements of the drawings are not necessarily to scale relative to each other, emphasis instead being placed upon clearly illustrating the principles of the disclosure. Furthermore, like reference numerals designate corresponding parts throughout the several views.

FIG. 1 depicts an illustration of a target-specific primer and a barcoded universal primer.

FIG. 2 depicts an illustration of projected resolution comparison for PCR (one loci), conventional MLST (5-7 loci), multiplex barcoding amplification (50 to >100 loci) and WGS.

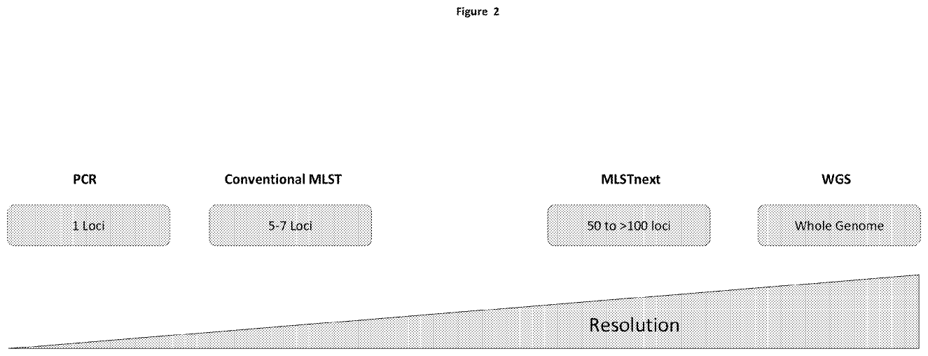

FIG. 3 depicts an illustration of the method for multiplex barcoding PCR, with a universal approach for a wide range of biological applications.

FIG. 4 illustrates the workflow of multiplex barcoding PCR.

FIG. 5 depicts an illustration of high-resolution multiplex barcoding amplification of 47 variable and housekeeping targets of Salmonella enterica.

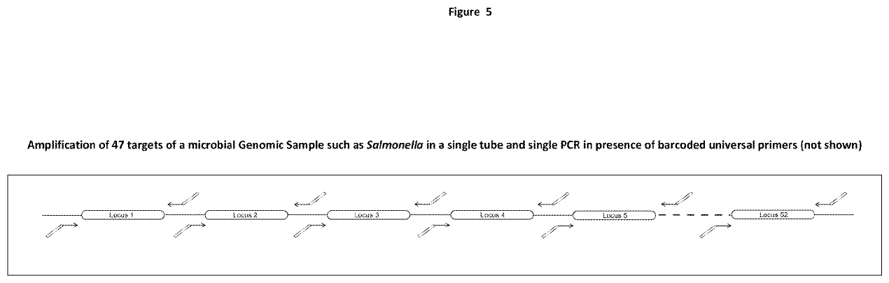

FIG. 6 depicts an illustration of 9 different Salmonella serotypes amplified by multiplex barcoding amplification in the same PCR tube/reaction and were differentiated by NGS.

FIG. 7 lists the pairwise matrix distances between tested Salmonella samples.

FIG. 8 shows Salmonella serotype calling from best-matched genome assembly.

FIG. 9 shows neighbor-joining tree for tested Salmonella samples.

FIG. 10 shows 3, 6 and 9 mixture of different Salmonella serotypes results from the same sample.

FIG. 11 shows the genotyping results for Cyclospora samples by analyzing 52 loci.

FIG. 12 shows the genotyping results for Legionella samples by analyzing 52 loci.

FIG. 13 shows the genotyping results for monkeypox samples by analyzing 21 loci.

FIG. 14 shows the genotyping results for Listeria samples by analyzing 69 loci.

FIG. 15 shows STEC serotype results by analyzing 61 loci.

FIG. 16 shows sequence read count for STEC serotype-specific wzx gene.

FIG. 17 shows sequence read count for other STEC gene markers.

FIG. 18 shows genotyping results for hepatitis A virus samples by analyzing 10 regions across HAV genome.

FIG. 19 shows the result of microbial identification for two environmental samples.

FIG. 20 shows genotyping results for HBV, HCV and HIV samples.

FIG. 21 shows mutations detected for breast cancer samples as well as the reference control BRCA Somatic Multiplex I (gDNA) control with allelic frequencies between 7.5% to 60%.

FIG. 22 shows STR insertions and/or deletions in forensic testing by multiplex barcoding amplification and NGS. The (−) symbol indicates deletion and (+) symbol indicates insertion. The number after either (+) or (−) indicates the number of copies of repeat unit detected in the assay. Two patterns are shown for each sample. All those insertions and/or deletions are related to hg19 genome reference.

### DETAILED DESCRIPTION

The present invention relates to a method of multiplex barcoding amplification, where amplification and barcoding occur simultaneously in the same PCR reaction (end product library) and then the barcoded amplicons are further analyzed by systems such as next-generation sequencing. The present invention has a universal approach and can be applied for biological application such as: (1) screening, detection and identification of bacteria/viral/parasitic/fungal and microbiome organisms; (2) detection, genotyping/subtyping and surveillance of bacteria/viral/parasitic/fungal by analyzing large number of variable/hypervariable sequences; (3) cancer/genetic disease nucleic acid sequence analysis; (4) pharmacogenetics and companion diagnostics for selecting the right treatment as well as prognosis; (5) drug resistance for applications such as antimicrobial therapy selection, surveillance and epidemiology, and personalized medicine; (6) forensic DNA analysis for DNA profiling; or (7) any biological application that uses multiplex barcoding amplification.

The present invention can also measure the DNA sequence variations in the selective regions and characterizes the species by their unique allelic profiles. The present invention provides methods, compositions, kits, systems, and instruments that will allow such target enrichment.

For microbial and viral genotyping/subtyping and surveillance, the nucleic acid differences of multiple variable loci are analyzed to determine genotype/serotype/strain/sub-strain level including tracing the source of infection (surveillance). This invention measures the DNA sequence variations in the selective loci and characterizes the species by their unique allelic profiles. The present invention provides methods, compositions, kits, systems, and instruments that will allow such target enrichment. The following examples, applications, descriptions and content are exemplary and explanatory, and are non-limiting and non-restrictive in any way.

Conventional MLST is a combination of PCR, sequencing and data analysis for the typing of multiple loci, using DNA sequences variable regions of an organism genome to characterize isolates of microbial, viral, fungal or parasitic species/subspecies. The application of MLST is wide-ranging, and provides a resource for the scientific, public health, and veterinary communities as well as the food industry. The advent of next generation sequencing technologies has made it possible to obtain sequence information across the entire microbial and viral genomes at relatively modest cost and effort. However, due to the sequence conservation in housekeeping genes, conventional MLST, which targets housekeeping genes sometimes lacks the discriminatory power to differentiate microbial and viral species to strain and sub-strain level, which limits its use in epidemiological investigations. Serological typing approaches have been established earlier for differentiating bacterial isolates, but immunological typing has drawbacks such as reliance on few antigenic loci and unpredictable reactivities of antibodies with different antigenic variants. Several molecular typing schemes have been proposed to determine the relatedness of pathogens such as pulsed-field gel electrophoresis (PFGE), ribotyping, and PCR-based fingerprinting. But these DNA banding-based subtyping methods do not provide meaningful evolutionary analyses. Despite PFGE being considered by many researchers as the “gold standard”, many strains are not typable by this technique due to the degradation of the DNA during the process (gel smears).

Multiplex barcoding amplification of a large number of variable/hypervariable sequences a microorganism in a single-tube, single-step reaction in conjunction with NGS-based MLST significantly enhances the resolution of microbial and viral species to strain and sub-strain level and will allow tracking the source of infection (surveillance).

Target-enrichment methods selectively capture genomic regions from a DNA sample before sequencing reaction through several steps. The drawback with existing target enrichment approaches is presence of multiple steps in their workflow, which increases time, cost and labor. In existing NGS workflows, amplification is performed in one or two rounds, and barcoding/indexing of each sample is performed in a separate step, which makes automation more challenging and may also increase the risk for cross-contamination. In conventional MLST, about 5-7 housekeeping genes are commonly used in order to strike the balance between the acceptable identification power, time and cost for the strain typing. Although the conventional MLST provides relatively acceptable discriminatory power, strain mistyping is not uncommon and obtaining sub-strain/subtype information is not achievable due to its low-resolution discriminatory power, which is critical for tracing and tracking the source of infection (microbial and viral surveillance).

The present invention comprises of a one-step, single-tube barcoding multiplex amplification step, with the ability to amplify a wide range of informative variable target sequences. In the PCR reaction, amplification and barcoding/indexing of nucleic acid targets occur simultaneously followed by NGS and data analysis. For microbial and viral typing/subtyping and surveillance, the analysis consists of: (1) data collection; (2) data analysis; and (3) multilocus sequence analysis. In the data analysis step, all unique sequences are assigned allele numbers and combined into an allelic profile and assigned a sequence type and the relatedness of strains/sub-strains are made by comparing allelic profiles. Based on the organism, a large set of data is generated during the sequencing and the generated sequence data are arranged, managed, analyzed and merged by bioinformatic tools.

In the data analysis, the generated sequences are assigned as alleles. The alleles at the loci provide an allelic profile. A series of profiles can then be the identification marker for typing to strain/sub-strain level. Sequences that differ at even a single nucleotide are assigned as different alleles and no weighting is given to take into account the number of nucleotide differences between alleles, as it cannot be distinguished whether differences at multiple nucleotide sites are a result of multiple point mutations or a single recombinational exchange.

In one aspect, the method and composition disclosed herein is designed to analyze target nucleic acids in a sample that is analyzed for disease (cancer/genetic disorders), drug resistance, genetic profile, forensics or microbial and viral organisms (bacteria, fungi, parasites or viruses). In some embodiments the disclosed method comprises the steps of: (1) contacting a set of variable nucleic acid targets in a sample with primers, wherein forward strand and reverse strand target-specific primers hybridize to different target regions in the test reaction; (2) amplifying the target nucleic acids under optimal amplification conditions to determine presence or absence of target nucleic acid; (3) sequencing the amplified products by NGS; and (4) analyzing the generated sequence reads.

In some embodiments, the present invention uses a mapping-and-counting method for microbial and viral typing, subtyping, and surveillance. In some embodiments, the mapping-and-counting method steps are as followed: (1) the score for a locus A for a candidate genome X is defined as the ratio between the number of unique reads mapped onto genome X's locus A and the total number of unique reads mapped onto the A locus for all analyzed genomes. If no read is mapped to genome X's locus A, the score is preset as a de minimis number, such as 1e⁻⁷; (2) the score for genome X is defined as the multiplication of scores for all tested loci; (3) Step (a), find the highest-scored genomes on remaining reads (at the beginning of the procedure, remaining reads mean all reads); if the number of non-empty loci for highest-scored genome is less than a preset cutoff, stop. In some embodiments, the preset cutoff is at least 10. Then, step (b), output the highest-scored genomes. Step (c), remove reads mapped to all highest-scored genomes thus far; and thereafter repeat step (a) to step (c). In some embodiments, this mapping-and-counting method may be performed using software.

Developing highly multiplex amplification methods for nucleic acid sample with accurate and high copy number sensitivity remains a challenge in the art. The present disclosure relates to an NGS-based assay that combines balanced target-specific multiplex amplification and sensitive copy number quantification, and balanced sequencing reads of each target while the barcoding of each target amplicon occurs simultaneously in the amplification step.

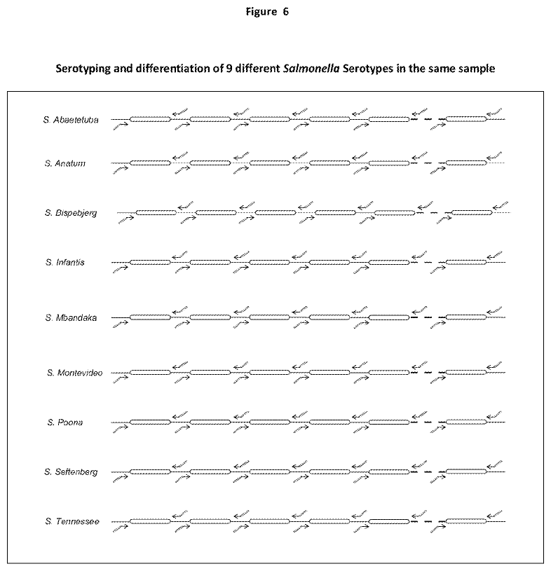

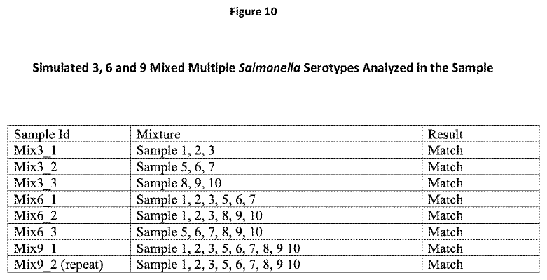

In another embodiment, for microbial and viral typing and subtyping, the present disclosure has the ability to detect and differentiate more than one serotype/genotype/strain/sub-strain in the same sample (FIGS. 6 and 10). The figure depicts detection of mixed strains of 3, 6 and 9 Salmonella serotypes in one sample.

All scientific terms used herein have the same meaning as commonly used and understood by one of ordinary skill in the art. Examples, materials, methods, figures and tables are illustrative only and not intended to be limiting.

As used herein, “amplification conditions” means conditions suitable for amplification using polymerase chain reaction. The polymerase chain reaction can be multiplex PCR. Amplification conditions include, but are not limited to, the examples provided in Examples 1-6 disclosed herein.

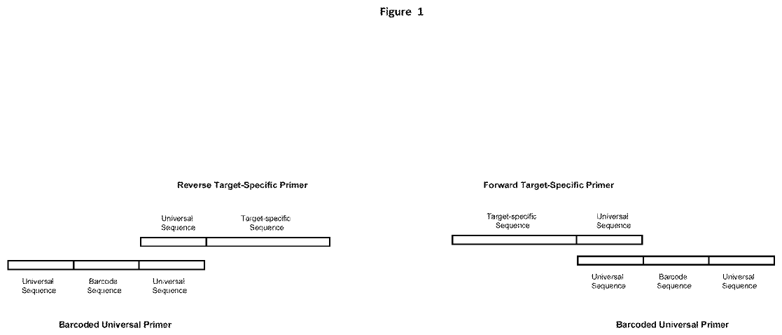

As used herein, “barcoded universal primer” means a universal primer comprising a barcode sequence and at least one universal sequence. See, e.g., FIG. 1.

As used herein, “bead cleanup” means the use of bead-based purification wherein beads are configured to bind to one or more targets. As known to those of skill in the art, bead cleanup may use positive selection (i.e., the bead is configured to capture the target of interest) or negative selection (i.e., the bead is configured not to capture the target of interest). Various may be used, as known in the art, such as streptavidin beads or magnetic beads.

As used herein, “compatibility score” means a score for a potential forward strand target-specific primer or reverse strand target-specific primer that is calculated based on different factors of target amplicon GC content, target amplicon melting temperature, target amplicon heterozygosity rate, complementary rate of the candidate primer for the target region; candidate primer size, target amplicon size, primer-primer interactions and amplification efficiency and off-target rate.

As used herein, “de minimis number” means a number equal or less than 1e⁻⁴ and preferably about 1e⁻⁷.

As used herein, “dsDNA” means double stranded DNA.

As used herein, “environmental source” means any potential location in a natural and/or man-made environment from which a sample can be taken. Environmental sources include but are not limited to: water sources such as oceans, lakes, ponds, rivers and streams; sources of soil such as soil, sand, internal or external dust; sources of gas, such as air.

As used herein, “FNA” means fine needle aspiration.

As used herein, “forward strand” means one strand of a dsDNA sample.

As used herein, “forward strand target-specific primer” means a primer configured to bind to a target sequence on the forward strand.

As used herein, “GC content” means guanine-cytosine content.

As used herein, “locus” means a specific physical position or location on the genome where a particular gene or genetic marker is located.

As used herein, “microbial and viral surveillance” means systematic monitoring and tracking of bacterial and viral pathogens in populations or specific geographical areas. It involves the collection, analysis, and reporting of data related to the occurrence, distribution, and characteristics of bacterial and viral infections.

As used herein, “Multilocus Sequence Typing (MLST)” means a technique that is used to classify microorganisms based on their genetic sequence at several different conserved genes. By analyzing the DNA sequences of several conserved genes, the genetic relatedness of different strains can assign the microorganism to a specific type. The information can be used to study the spread of microbial infections, track the evolution of microbial populations over time and identify genetic factors that may contribute to virulence or antibiotic resistance.

As used herein, “multiplex barcoding amplification” means multiplex target amplification where amplification and indexing/barcoding of each target occurs simultaneously in the PCR reaction.

As used herein, “NGS” means next-generation sequencing.

As used herein, “PCR” means polymerase chain reaction.

As used herein, “reverse strand” means a second strand of a dsDNA sample that is complementary to the forward strand.

As used herein, “reverse strand target-specific primer” means a primer configured to bind to a target sequence on the reverse strand.

As used herein, “sample” means a specimen or a preparation in the field to which the present invention pertains. Samples may be obtained from various sources, such as subjects, food, plants, and environmental sources. In case where the term is used in the present specification with respect to a subject, for example, the “sample” means a “biological sample” or an equivalent thereof. The “biological sample” means any preparation obtained from a biological material (e.g., individual, liquid, body fluid, cell line, cultured tissue or tissue segment) serving as a source. Examples of the “biological sample” include body fluids (e.g., blood, saliva, dental plaque, blood serum, blood plasma, urine, synovia, and cerebrospinal fluid) and tissue sources.

As used herein, “species” means a group of microorganisms that share similar genetic and phenotypic characteristics.

As used herein, “species specific primer” means a primer configured to bind to a target specific to a particular species.

As used herein, “strain” means is a subgroup of bacterial species that has unique genetic and phenotypic characteristics that distinguish it from other members of the same species. Strains can differ in their virulence, antibiotic resistance, or other traits that affect their behavior in the environment or their interactions with other organism.

As used herein, “sub-strain” means a further subdivision of a bacterial strain that has additional genetic or phenotypic differences from other members of the strain. Sub-strains are typically identified by additional genetic markers or phenotypic traits.

As used herein, “subject” means an animal, preferably a mammal, and most preferably a human.

As used herein, “subtype” means to further classify microorganisms and viruses within a specific type. In microbiology, a bacterial subtype may be identified based on specific genetic or phenotypic characteristics, such as antibiotic resistance or virulence factors. In virology, a viral subtype may be identified based on specific genetic mutations or changes in antigenic properties.

As used herein, “target-specific primer” means a primer configured to bind to a specific target. In some embodiments, a target-specific primer may be a type-specific primer. In some embodiments, a target-specific primer may be a species-specific primer. In some embodiments, a target-specific primer may be a specific sequence/region on a microbial or viral genome.

As used herein, “type” in microbiology means to refer to the strain or specific type of a microorganism. In virology, it means classification of viruses based on their genetic and antigenic characteristics.

As used herein, “type-specific primer” means a primer configured to bind to a target specific to a particular microbial or viral genome.

As used herein, “universal sequence” means a sequence configured to be targeted by a universal sequence primer.

The present disclosure describes methods, compositions and kits for amplification and enrichment of specific and known sequence nucleic acid targets for determining the nucleotide sequence. The following examples, applications, descriptions and content are exemplary and explanatory, and are non-limiting and non-restrictive in any way.

The present disclosure relates to selective amplification of a set of target sequences by multiplex barcoding amplification and further analysis by next generation sequencing. The disclosure has universal approach for a wide range of biological applications.

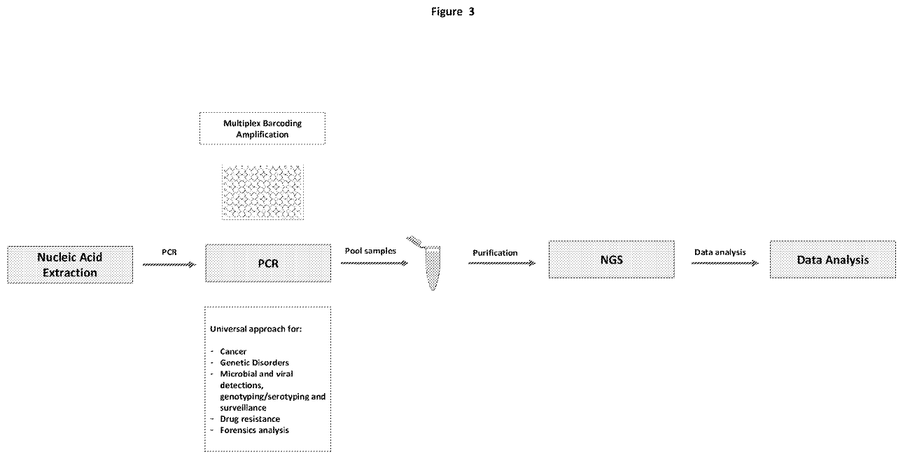

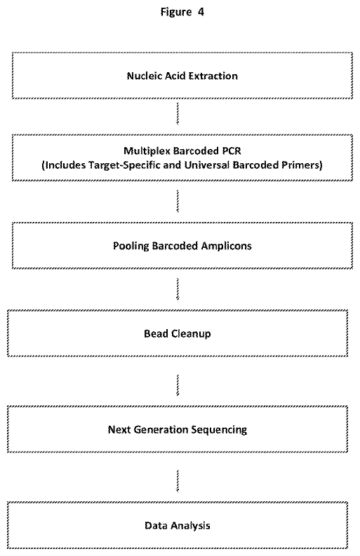

The disclosed method offers many advantages that are uniquely composed together including, but not limited to: (1) single reaction PCR; (2) one-round of highly multiplex PCR covering a wide range of targets; (3) dual-index barcoding (barcodes on both ends of each amplicon) to minimize cross-contamination; (4) target-specific amplification allowing uniform amplification; (5) quantification of target nucleic acid; (6) an internal control for each sample in the test reaction to monitor amplification, which may serve as a normalization factor for quantification; (7) suitable for high throughput scales; (8) the method is applicable to a biological applications for multiplex barcoding amplification; and (9) simple workflow and easy automation. See, e.g., FIGS. 3-4.

While WGS analyzes the entire genome, a focused target-specific panel continues to offer the advantages of better coverage of targeted regions, greater facility to detect multiple variant types, substantially at lower costs, higher throughput, simpler bioinformatics analysis, and focused testing, obviating the need to deal with secondary/incidental findings that otherwise inevitably arise with whole genome sequencing. Furthermore, targeted sequencing of specific regions of interest in a large number of samples is much more cost-effective in providing answers to biological questions than sequencing the whole genomes of fewer organisms. An efficient and specific target enrichment method allows more efficient targeted sequencing. The important parameters for target enrichment are: (i) sensitivity, (ii) specificity, (iii) uniformity, (iv) reproducibility, (v) cost, (vi) ease of use, and (vii) amount of DNA required per experiment (Nat Methods. 2010 February;7(2):111-8). A multiplex barcoding amplification (where both amplification and barcoding take place simultaneously in the PCR reaction) can be applied to a wide range of biological applications (FIG. 3).

Herein described is a method of multiplex amplification and target enrichment, which are analyzed by NGS. In various embodiments of any of the aspects of the present disclosure, the methods and compositions feature multiplex amplification and target enrichment of target nucleic acid regions of genomic material such as cancer, genetic disorders, drug resistance, forensics and microbial and viral organisms.

In some embodiments, the present invention is applied for detection, identification, genotyping, and typing to strain and sub-strain level. In some embodiment, the disclosed method comprises the steps of: (1) contacting target-specific primers with a set of variable/hypervariable target nucleic acid sequences across a microbial or viral genome in the presence of barcoded universal primers and hybridizing to target nucleic acid sequences in the sample; (2) subjecting the test reaction to amplification under optimal amplification conditions; (3) pooling together the amplified products from each individual or subject sample; (4) a portion of the pooled amplified products are subjected to bead cleanup to remove possible unconsumed primers and primer-dimers to create enriched amplified products; (5) subjecting a portion of enriched amplified products to standard normalization and quantification; and (6) sequencing the amplicon by next-generation sequencing. See, e.g., FIGS. 3-4.

In some embodiments, the present invention is applied for nucleic acid sequence analysis of target regions for cancer, genetic disorders, forensics, pharmacogenetics, and drug resistance. In some embodiment, the disclosed method comprises the steps of: (1) contacting target-specific primers to target nucleic acid sequences in a sample in the presence of barcoded universal primers and hybridizing to target nucleic acid sequences in the sample; (2) subjecting the test reaction to amplification under optimal amplification conditions; (3) pooling together the amplified products from each individual or subject sample; (4) a portion of the pooled amplified products are subjected to bead cleanup to remove possible unconsumed primers and primer-dimers to create enriched amplified products; (5) subjecting a portion of enriched amplified products to standard normalization and quantification; and (6) sequencing the amplicon by next-generation sequencing. See, e.g., FIGS. 3-4.

In one embodiment, the barcoded universal primers comprise: (a) a universal priming portion at the 3′-end; (b) a barcode portion in the middle; and (c) a universal priming portion at the 5′-end (FIG. 1). In one embodiment, each target-specific primer comprises a specific sequence portion directed to target nucleic acid sequence and a universal priming portion.

In some embodiments, the present disclosure provides methods, compositions and kits to detect more than one genotype/serotype/strain/sub-strain/type/subtype in a single reaction container, such as a single tube (See, e.g., FIG. 6). The number of genotypes/serotypes/strains/sub-strains/types/subtypes in the same sample can be 2, 3, 4, 8, 12, 16, 20, 30 or more.

In some embodiments, the disclosed method utilizes one round of multiplex PCR in one single test reaction for each subject, which minimizes DNA cross-contamination and extra steps in the workflow. In general, methods using more than one round of PCR are vulnerable to DNA cross-contaminations and may cause inaccurate results.

In some embodiments, the disclosed method is a highly multiplex microbial and viral identification panel, covering an unprecedented and unparalleled wide range variable/hypervariable regions (including housekeeping genes) within a microbial and viral genome in a single reaction and one round of PCR.

In some embodiments, the disclosed method comprises the use of a housekeeping gene or a control gene in a microbial and viral genome as internal control for each subject in each test reaction, which may monitor amplification and is used as normalization factor for quantification of copy number of microbial and viral genome for each sample in each test reaction. In some embodiments, the method can quantify multiple strains/sub-strains in a multiple co-infected sample in one test reaction.

In some embodiments, the disclosed method comprises the use of a dual barcoding index, wherein the amplicon is barcoded by universal barcoded primers on both ends, minimizing cross-contamination and as well as dual confirmation of a barcode in case of amplification errors in early stages of PCR.

In some embodiments, the disclosed method comprises the use of next-generation sequencing for screening, detection, identification, and quantification of microbial or viral genomes. In certain embodiments, target nucleic acid sequences are amplified and sequenced to reveal the strain or sub-strain present in a sample, which could be used for tracing the source of infection (microbial and viral surveillance). In some embodiments, the disclosed method comprises the use of next-generation sequencing for analyzing target nucleic acid regions of genomic material of samples related to cancer/genetic disorders, drug resistance, forensics and other biological applications.

In some embodiments, the amplification conditions such number of cycles, annealing temperature, annealing duration, extension temperature and extension duration are adjusted to optimal conditions for amplification. In some embodiments, number of cycles, the amplification conditions such annealing temperature, annealing duration, extension temperature and extension duration are adjusted to optimal conditions for amplification based on the commercial DNA polymerase instructions.

In some embodiments, the nucleic acid sample comprises genomic DNA or RNA. In another embodiment, the sample comprises nucleic acid molecules obtained fresh produce, food, imported food, food production facilities, farms, fresh produce, animal farms, water, spoilage, soil and environment. In another embodiment, the sample comprises nucleic acid molecules obtained from swab or brush. In some embodiments, the sample comprises nucleic acid molecules obtained from saliva. In some embodiments, the sample comprises nucleic acid molecules obtained from urine, tissue, saliva, biopsies, sputum, swabs, formalin-fixed paraffin-embedded material (FFPE), surgical resections, cervical swabs, tumor tissue, fine needle aspiration (FNA), scrapings, swabs, mucus, urine, semen, and other non-restricting clinical or laboratory obtained samples.

In some embodiments, the nucleic acid sample obtained can be from an animal such as a human or mammalian subjects. In another embodiment, the nucleic acid sample obtained can be from a non-mammalian subject such as bacteria, parasites, virus, fungi, and plant.

In some embodiments, the disclosure relates to target amplification of at least one target sequence from a biological sample in a normal or diseased subject. In some embodiments, the disclosure relates to the specific and selective target amplification of at least one target sequence and detection and identification of microbial and viral species/strains/sub-strains in the nucleic acid sample.

In some embodiments, the target-specific primers comprise a plurality of primers that are designed to amplify selectively variable and hypervariable regions of microbial and viral target nucleic acid sequences; the amplification range differs due the size of fragments and positions of primers on the nucleic acid fragment and the size can vary in the range. In some embodiments, the target-specific primers comprise a plurality of primers that are designed to amplify selectively target nucleic acid sequences of genomic material of samples related to cancer, genetic disorders, drug resistance, forensics, and other biological applications.

The amplification range differs due the size of fragments and positions of primers on the nucleic acid fragment and the size can vary in the range. In some embodiments, the target-specific primers comprise a plurality of primer that are selectively designed to amplify target nucleic acid sequences, where the amplified target nucleic acid sequences can vary in length from one another by no more than 90%, no more than 70%, no more than 50%, no more than 25% or no more than 10%.

In some embodiments, the disclosed method relates to target enrichment by multiplex target-specific PCR, which comprises the steps of contacting the nucleic acid targets with a plurality of target-specific primers in the presence of barcoded universal primer and PCR reagents such as DNA polymerase, dNTPs and reaction buffer; given the optimal conditions of temperature and time for denaturation, annealing and extension, the primers hybridize to complementary target nucleic acid sequences and are extended. In some embodiments, the amplification steps can be performed in any order. In some embodiments, amplification steps, purification steps and cleanup steps could be added or removed upon optimization for optimal multiplex target amplification for downstream processes.

In some embodiments, the described method uses PCR and DNA polymerase as one of the components in the reaction. In some embodiments, there are a wide selection of DNA polymerases, which feature different characteristics such as thermostability, fidelity, processivity and Hot Start. The method can use a DNA polymerase with one or more of these features depending on the application. In some embodiments, the concentration of DNA polymerase for multiplex PCR can be higher than single-plex PCR.

In some embodiment, the method disclosed herein uses amplification of target nucleic acid sequences using multiplex polymerase chain reaction, wherein more than one target sequence is amplified in a test reaction. In some embodiments, the amount of nucleic acid sample needed for multiplex amplification can be about 0.1 ng. In some embodiments, the amount of nucleic acid material can be about 1 ng, 5 ng, 10 ng, 50 ng, 100 ng or 200 ng.

In some embodiments, the disclosed method herein uses amplification of target nucleic acid sequences using multiplex polymerase chain reaction, wherein more than one target sequence is amplified in a test reaction. The state-of-art polymerase chain reaction is performed on a thermocycler and each cycle of PCR comprises of denaturation, annealing and extension. Each cycle of PCR comprises at least denaturation step, one annealing step and one extension step for extension of nucleic acids. In some embodiments, annealing and extension can be merged. In some embodiments, the method disclosed herein comprises 25 to 35 cycles of PCR. Each cycle or set of cycles can have different durations and temperatures, for example the annealing step can have incremental increases and decreases in temperature and duration, or the extension step can have incremental increases and decreases in temperature and duration. In some embodiments, duration can have decreases or increases in 5 seconds, 10 seconds, 30 seconds, 1 minute, 2 minutes, 4 minutes, 8 minutes, or greater increments. In some embodiments, temperature can have decreases or increases in 0.5, 1, 2, 4, 8, or 10° Celsius increments.

In some embodiments of the present disclosure, the target-specific primers comprise a nucleotide modification in 3′-end or 5′-end or across the sequence. In some embodiments, the length of target-specific portion of the primer can be 15 to 40 bases. In some embodiments, the Tₘ of each target-specific primer can be about 55° C. to about 72° C.

In some embodiments, the disclosure features a target enrichment and multiplex amplification approach for target specific nucleic acid amplification of microbial and viral species/strains/sub-strains using target-specific primers. In some embodiments, the disclosure features a target enrichment and multiplex amplification approach for target specific nucleic acid amplification of genomic material related to cancer/genetic disorders, drug resistance, forensics and other biological applications. In some embodiments, the selected target-specific primers contact and hybridize to target nucleic acid sequences that can be related to disease. In one embodiment, target-specific primers hybridize to nucleic acid sequences in the test reaction, which have different sizes. In some embodiments, amplicon size selection can be used to sequence amplified products of a certain length range. In some embodiments, amplicons of 100 to 250 base pairs range in length can be sequences. In some embodiments, amplicons of 150 to 300 base pairs, or amplicons of 120 to 350 base pairs, or amplicons of 200 to 500 base pairs range or greater length range can be sequenced.

In some embodiments, any of the procedures can be removed or can be repeated. In some embodiments, purification steps can be added for generating optimal results. These procedures are non-limiting and a skilled person of the art can readily add, remove or repeat the steps for optimal results.

The ability to increase the number of target-specific primers in a multiplex PCR allows simultaneous amplification of a large number of nucleic acid targets while decreasing the amount of input DNA, labor and time. This is especially advantageous when the amount of starting input nucleic acid material is limited.

In some embodiments of the disclosed method, the primer design methodology selects the candidate target-specific primers based on this stepwise procedure: (1) extraction of genomic sequence around each targeted variant position; (2) for each variant in the target sequence, design target-specific forward strand and reverse strand target-specific primers with proper GC content, Tₘ, and varying distances from each targeted variant; (3) for each primer, searching target genome sequences for off-target matches; filter primers and keep those primers that pass the off-target threshold; (4) search the 3′-end portion of each primer for complementary matches with primer sequences of the set; filter primers progressively where the primer with its 3′-end having most complementary matches is removed first; (5) synthesize primers and run the entire wet-lab experiment comprising next-generation sequencing; calibrate the performance of each primer and filter out primers of undesired performance. In some embodiments, the primer selection procedure steps 2 to 4 and steps 2 to 5 are repeated until each target variant is covered by at least one forward strand target-specific primer and one reverse strand target-specific primer in the primer set.

In some embodiments, the disclosure features a primer design methodology that eliminates low compatibility primers that form artifacts such as primer-dimers in a highly multiplexed PCR that inhibit efficient amplification. Such elimination system removes or significantly minimizes the non-productive artifacts such as primer-dimers. Removal of low-compatibility and problematic primers significantly improves the overall performance and efficiency of highly multiplex PCRs in addition to downstream processes such as high throughput sequencing. Artifacts and primer dimers cause significant failure in obtaining optimal sequence results and a significant portion of the sequencing reads can be non-specific and non-informative.

In some embodiments, the primer selection methodology features a primer compatibility score both in regard to primer-primer interactions and specific target nucleic acid hybridization without non-specific priming or hybridizing to off-target regions. A higher compatibility score for a candidate target-specific primer characterizes specific hybridization to target nucleic acid with no or minimal interaction with other primers in the primer set. Primers that do not meet the compatibility score that is to say are above the minimum threshold are removed. In various embodiments of the disclosed method, a compatibility score is calculated for at least 80, 90, 95, 98, 99, or 99.5% of the possible combinations of candidate primers in the set. The compatibility score in primer selection is calculated based on a number of parameters such as target amplicon GC content, target amplicon melting temperature, target amplicon heterozygosity rate, complementary rate of the candidate primer for the target region, candidate primer size, target amplicon size and amplification efficiency. Due to the fact that several aspects are involved in determining the compatibility score, an average score is calculated based on multiple parameters and average could be variable for particular applications. The primer selection methodology will keep eliminating the low-compatibility primers, and the elimination process is repeated to equal or below minimum threshold till an optimal selection primer group is achieved that generates a highly multiplex target amplification PCR with no or minimized primer-dimers.

In some embodiments, the primer selection methodology features a primer compatibility score both in regard to primer-primer interactions and specific target nucleic acid hybridization without hybridizing to off-target regions. The primers that have low compatibility score that is to say above the minimum threshold will be eliminated. However, if there are limitations in primer selection in certain applications, the minimum threshold can be increased to a higher level of second threshold to facilitate primer selection for the primer group. In some embodiments the selection process is repeated until candidate primers are selected that are equal or under the second level of minimum threshold.

In an embodiment, the disclosed method herein features a multiplex amplification and target enrichment by utilizing target-specific primers that contact target nucleic acid sequences of genomic material of samples related to cancer/genetic disorders, drug resistance, forensics, microbial, fungal, parasitic, viral and other biological applications, wherein primer dimers can be reduced or minimized by adjusting different parameters such as duration of annealing steps, increase or decrease of temperature increments combined with number of cycles. In some embodiments, the primer concentrations can be lowered, and annealing temperature and duration can be increased to allow specific amplification (the primers have more time interval to hybridize to target nucleic acids) in addition to reduced or minimal primer-dimers. In some embodiments, the concentration of primers can be 500 nM, 250 nM, 100 nM, 80 nM, 70 nM, 50 nM, 30 nM, 10 nM, 2 nM, 1 nM or lower than 1 nM. In some embodiments, the annealing temperature could be 1 minute, 3 minutes, 5 minutes, 8 minutes, 10 minutes or longer. In some embodiments, the amplification with longer annealing time uses 1 cycle, 2 cycles, 3 cycles, 5 cycles, 8 cycles, 10 cycles or more followed by standard annealing durations.

In one aspect, the disclosed method comprises the step of amplifying selective target nucleic acid sequences of samples related to cancer/genetic disorders, drug resistance, forensics, microbial, fungal, parasitic, viral and other biological applications. In some embodiments, the method comprises the step of contacting the nucleic acid sample with target-specific primers in presence of barcoded universal primers in a test reaction. In some embodiments, the method comprises the step of determining the presence or absence of target amplification product. In some embodiments, the method comprises the step of determining the sequence of the amplified target products. In some embodiments, the method identifies the microorganism to strain or sub-strain level. In some embodiments, less than 50, 40, 30, 20, 10, 5, 0.5, or 0.1% of the amplified products are primer-dimers or artifacts. In one embodiment, there can be more than one set of target specific primers as an example there can be two sets of target-specific primers for two test reactions, 3 sets for 3 test reactions or 5 sets for 5 test reactions or more. In some embodiments for practical reasons such as limitations in primer design or selection, the sample may also be split into multiple parallel multiplex test reactions with multiple sets of target-specific primers.

In various embodiments, concentration of each primer can be 500 nM, 250 nM, 100 nM, 80 nM, 70 nM, 50 nM, 30 nM, 10 nM, 2 nM, 1 nM or lower than 1 nM. In various embodiments, primer concentration of each primer can be between 1 μM and 1 nM, between 1 nM and 80 nM, between 1 nM and 100 nM, between 10 nM and 50 nM or 1 nM and 60 nM. In some embodiments, the GC content of target-specific primers can be between 40% and 70%, or between 30% and 60% or 50% and 80% or 30 and 80%. In some embodiments, primer GC content range can be less 20%, 15%, 10% or 5%. In some embodiments, the melting temperature (Tₘ) of the target-specific primers can be between 55° C. and 65° C., or 40° C. and 72° C., or 50° C. and 68° C. In some embodiments, the melting temperature range of the primers can be less 20° C., 15° C., 10° C., 5° C., 2° C. or 1° C. In some embodiments, the length of the target-specific primers can be between 20 and 90 bases, 40 and 70 bases, 20 and 40 bases or 25 and 50 bases. In some embodiments, the range of length of the primers can be 60, 50, 40, 30, 20, 10, or 5 bases. In some embodiments, the 5′-region of the target-specific primer is a universal priming site that are not complementary or specific for any target nucleic acid regions.

In one aspect, the present disclosure is directed to a kit that comprises target-specific primers in a group; the primers are designed and selected based on criteria described to have minimal primer-primer interactions or non-specific priming. In another embodiment, the kit can be formulated for detection, screening, diagnosis, prognosis and treatment of disease. In another embodiment, the kit can be formulated for detection of drug resistance. In some embodiment, the kit can be used for bacterial, fungal, parasite and viral screening, detection, identification, genotyping, subtyping, and surveillance. In some embodiment, the kit can be used for analysis of samples related to cancer/genetic disorders, forensics, drug resistance, pharmacogenetics and other biological applications.

In some embodiments, the disclosed method comprises the steps of: (1) contacting a set of target-specific primers with target nucleic acid sequences in the presence of barcoded universal primers and hybridizing to target nucleic acid sequences in each sample in the test reaction; (2) subjecting the test reaction to amplification under optimal amplification conditions; (3) pooling together the amplified products from each individual sample; (4) subjecting a portion of the pooled amplified products to bead cleanup to remove possible primer-dimers to create enriched amplified products; (5) subjecting a portion of the enriched amplified products to standard normalization and quantification; and (6) sequencing the amplicon by next-generation sequencing. The method may further comprise additional steps, such as purification. In one aspect, highly multiplex PCR is utilized for the method disclosed. In some embodiments, between 1 and 10 cycles of PCR can be performed for PCR; in some embodiments between 1 and 15 cycles or between 1 and 20 cycles or between 1 and 25 cycles or between 1 and 30 cycles, between 1 and 35 cycles or more can be performed.

In another embodiment, the disclosed method can be used in a multiplex fashion when amplifying more than two targets and is not limited to any number of multiplexing.

In some embodiments, the amplification product can be sequenced by next-generation sequencing platforms. Next-generation sequencing is referred to non-sanger based massively parallel DNA nucleic acid sequencing technologies that can sequence millions to billions of DNA strands in parallel. Examples of current state of state-of-art next-generation sequencing technologies and platforms are Illumina platforms (reversible dye-terminator sequencing), 454 pyrosequencing, Ion Semiconductor sequencing (Ion Torrent), PacBio SMRT sequencing, Qiagen GeneReader sequencing technology, Element Biosciences Sequencing platforms, and Oxford Nanopore sequencing. The present disclosure is not limited to these next-generation sequencing technologies examples.

### Example 1

### High Resolution Salmonella Genotyping and Subtyping

### Materials and Methods

Samples and DNA Extraction: For this study, 12 ATCC Salmonella isolates and three other isolates were used: S. abaetetuba, S. anatum, S. bispebjerg, S. diarizoniae, S. infantis, S. Mbandaka, S. Montevideo, S. Poona, S. Seftenberg, S. Tennessee, S. Typhi, and S. Typhimurium. Stocks of the bacterial strains preserved in glycerol were recovered from −80° C. stock storage. Isolates were streaked on Tryptic Soy Agar with 5% Sheep Blood plates and incubated at 35° C. overnight. One to three colonies were picked, and their DNA were extracted using the QIAamp Mini DNA kit following the manufacturer's protocol. Salmonella isolates were propagated, and DNA was extracted.

Assay Design: For Salmonella genotyping to sub-strain level, the assay was designed to examine 47 polymorphic loci from Salmonella enterica genome. The 47 loci include seven loci for housekeeping genes (aroC, dnaN, hemD, hisD, purE, sucA, and thr A) used in traditional MLST assays. Although the assay is designed based on S. enterica genome, more than 20 loci could get amplified and sequenced for Salmonella bongori.

Amplification: One-step multiplex PCR was performed in 15 μl final volume in a 96-well plate on a Veriti thermocycler (ThermoFisher, CA, USA). The PCR reaction consisted of target-specific primers, barcoded universal primers, sample DNA, DNA polymerase, dNTPs and PCR buffer.

Next-generation sequencing: All the amplicons were pooled into one tube. A portion of the samples was then purified with SPRIselect beads (Beckman Coulter, CA, USA) according to the manufacturer's instructions. The purified sample concentration was measured on a Qubit 3 and the concentration was normalized for sequencing. The library was sequenced with Illumina MiniSeq system using an Illumina Mid Output sequencing kit.

Sequence data analysis software and interpretation: Decoding: sequencing reads (FASTQ format) for forward and reverse reads and forward and reverse indexing reads were input in ChapterDx Analysis Software. The software assigns sequencing reads for a sample based on the sequence of both forward and reverse index reads with no mismatch; Mapping: sequencing reads are mapped onto reference sequences using the Smith-Waterman algorithm with options as nucleotide match reward is 1, nucleic mismatch penalty is −3, cost to open a gap is 5, and cost to extend a gap is 2. Only the alignment of best match is kept for each sequencing read if the alignment score is beyond 60. Subtyping: (1) find the genome (X) with most mapped loci; (2) exit if the number of mapped loci is less than pre-set cutoff; (3) output genome (X) and remove all mapped reads to genome (X); and (4) repeat step 1-3. In some embodiments, the preset cutoff is at least 10. For each locus, paired (one forward and one reverse) consensus sequences was generated. Pairwise distance was calculated as the number of allele difference between two strains. Neighbor-joining tree was then constructed based on the resultant distance matrix.

### Results

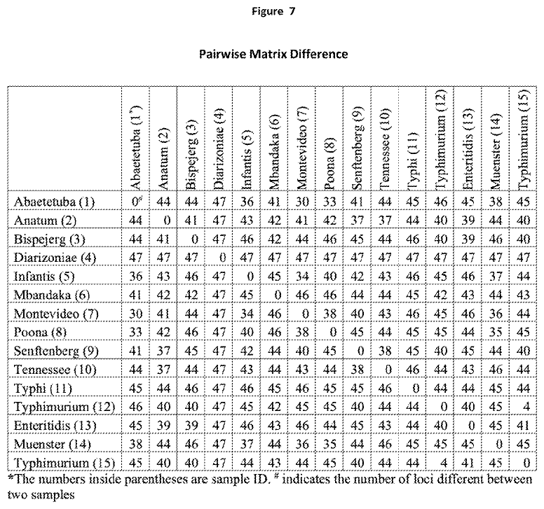

Distance between Samples: FIG. 7 lists the distances between each pair of tested samples. Pairwise distance was calculated as the number of allele difference between two strains, which is the same as traditional MLST. In traditional MLST the number of nucleotide differences between alleles is ignored and sequences are given different allele numbers whether they differ at a single nucleotide site or at many sites. The rationale is that a single genetic event resulting in a new allele can occur by a point mutation (altering only a single nucleotide site), or by a recombinational replacement (that will often change multiple sites). FIG. 7 shows isolates of different serovars differ more than 30 loci, suggesting that loci examined by multiplex barcoding amplification NGS assay are heterogenous. Sample 12 and 15 are both S. typhimurium serovar. However, alleles at four loci are different for these two samples of the same serovar. These four loci do not overlap any of the seven housekeeping genes (aroC, dnaN, hemD, hisD, purE, sucA, and thrA) used in traditional MLST. Thus, traditional MLST would have unlikely discriminated them.

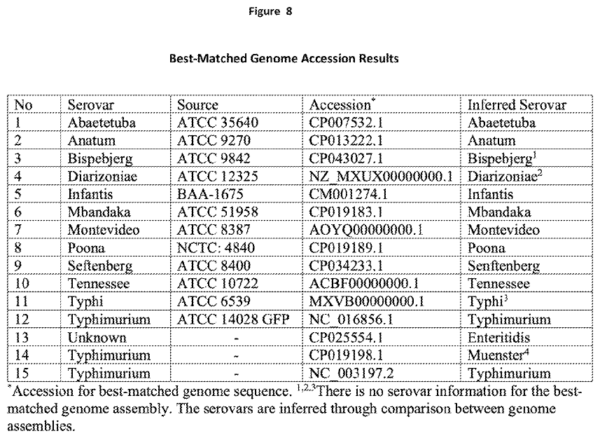

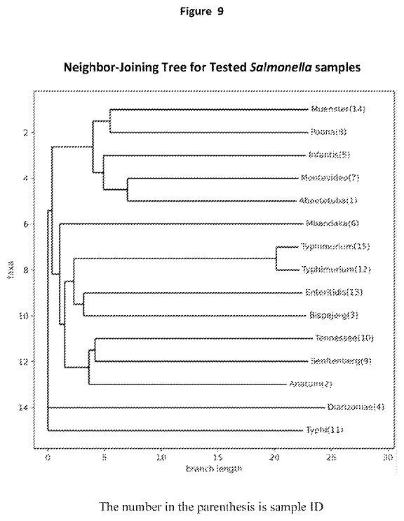

Genotyping: The genotype calling methodology infers the serovar from the best-matched genome assembly. Often, the best-matched genome assemblies are annotated with serovar information. However, sometimes, the best-matched genome assemblies lack serovar information. In order to fill the missing serovar for genome assemblies, several different methodologies have been tested to estimate the evolutionary distances among Salmonella assemblies downloaded from the NCBI database. Eventually, the program and was chosen to calculate the distance between two Salmonella genome assemblies because the resultant distance matrix reflected the relationships among Salmonella genomes. Based on the calculated distance matrix, many Salmonella genome assemblies without serovar information were grouped together with those with serovar information using the single-linkage clustering methodology. FIG. 8 lists the NCBI accessions for best-matched genome assemblies and their corresponding inferred serovars. There was no serovar information for sample 13 that the assay identified the serotype as S. enteritidis. Sample 14 was labeled as S. typhimurium originally, but our assay identified it as S. muenster with best-matched genome accession CP019198.1. The neighbor-joining tree (FIG. 9) based on allele difference shows that sample 14 is separated from both sample 12 (S. typhimurium) and sample 15 (S. typhimurium) with 45 loci difference FIG. 7, suggesting that sample 14 is less likely to be S. typhimurium.

Detecting co-presence of multiple strains in the same sample: It has been reported that the same food or environmental samples could be contaminated with different Salmonella strains simultaneously. As such multiple serotype Salmonella outbreaks might occur more frequently than recognized. Identification of all Salmonella serotypes involved in an outbreak might help implicate the outbreak source, define the scope of the outbreak, and determine the selection of appropriate control measures. To evaluate the assay's capability to detect co-presence of multiple strains in the same samples, DNA from multiple samples were mixed to mimic samples contaminated with multiple strains. The experiment was performed in mixtures of 3, 6 and 9 serovars, where (1) three tubes contained mixture of three serovars each, (2) three tubes contained mixture of six serovars each, and (3) two tubes contained mixtures of nine serovars each. The two mixture tubes with nine serovars were identical. The results show that the multiplex barcoding amplification NGS assay could identify and differentiate all serovar by ChapterDx Data Analysis Software from all mixtures, suggesting this assay's excellent capability to detect co-presence of multiple strains in the same sample (FIG. 10).

### Example 2

### High-Resolution Cyclospora Cayetanensis Genotyping

### Materials and Methods

Samples and DNA Extraction: DNA were extracted from a variety of fresh produce items including salad mix (romaine and iceberg lettuces, carrots, and red cabbage), cilantro, and basil samples (25 g each) inoculated with a preparation of purified C. cayetanensis oocysts from a patient. In addition, samples of 50 liters of pond water were inoculated with between 200 and 20,000 oocysts. Sample preparation and DNA extraction were done according to procedures described previously (Durigan, M., Murphy, H. R., and Da Silva, A. J. (2020). Dead-End Ultrafiltration and DNA-Based Methods for Detection of C. cayetanensis in Agricultural Water (Appl Environ Microbiol 86; Almeria, S., and Shipley, A. 2021). Detection of C. cayetanensis on bagged pre-cut salad mixes within their shelf-life and after sell by date by the U.S. food and drug administration validated method (Food Microbiol 98, 103802; Assurian, A., Murphy, H., Ewing, L., Cinar, H. N., Da Silva, A., and Almeria, S. 2020). Evaluation of the U.S. Food and Drug Administration validated molecular method for detection of Cyclospora cayetanensis oocysts on fresh and frozen berries. Food Microbiol 87, 103397).

Assay Design: Contigs for Cyclospora cayetanensis isolate NF1 (accession MSEL00000000) were concatenated as a reference genome from which fragments between 280 and 350 bases were obtained. Fragments were used as BLAST queries against the 40 WGS assemblies of C. cayetanensis, and fragments with BLAST hits in all 40 WGS assemblies were kept. The entropy was calculated for each fragment and fragments with entropy values>1.0 were considered for inclusion. Fragments evenly located along the artificial genome were then picked as templates for primer design. 52 loci were chosen for Cyclospora cayetanensis.

Mulitplex Barcoding Amplification: One-step multiplex barcoding PCR was performed using the Cyclospora NGS Assay. Briefly, for each sample, 5 μl DNA was used in a single multiplex PCR containing target-specific primers in the presence of barcoded universal primers, DNA polymerase, dNTP and PCR buffer for all amplicons.

Next-generation sequencing: All the amplicons were pooled into one tube. A portion of the samples was then purified with SPRIselect beads (Beckman Coulter, CA, USA) according to the manufacturer's instructions. The purified sample concentration was measured on a Qubit 3 and the concentration was normalized for sequencing. The library was sequenced with Illumina MiniSeq system using an Illumina Mid Output sequencing kit.

### Results

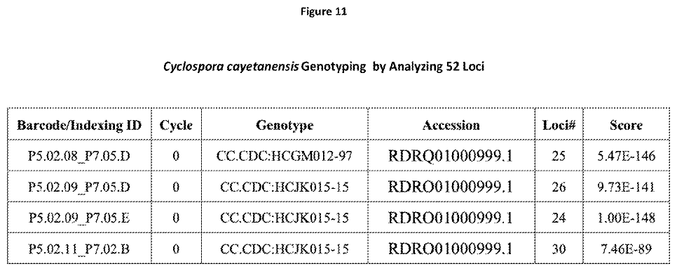

FIG. 11 show the genotype results for Cyclospora samples. The table lists the genotype results for Cyclospora samples. Although majority of targeted loci were amplified and sequenced, the best scored genome matches only roughly half of those loci. Nevertheless, this example shows the feasibility of this purposed technology on the Cyclospora genotyping.

### Example 3

### Legionella Genotyping by Multiplex Barcoding Amplification and Extensive Variable Multilocus Sequence Typing

### Materials and Methods

Samples and DNA Extraction: DNA were extracted from enriched 12 Legionella samples.

Assay Design: For Legionella genotyping to sub-strain level, the assay was designed to cover 55 polymorphic loci across Legionella pneumophila genome. The assay also detects through conservative gene targets other Legionella species such as L. longveachae and L. bozemanii.

Amplification: One-step multiplex barcoding PCR was performed on the extracted DNA by target-specific primers in the presence of barcoded universal primers, DNA polymerase, dNTP and PCR buffer.

Next-generation sequencing: All the amplicons were pooled into one tube. A portion of the samples was then purified with SPRIselect beads (Beckman Coulter, CA, USA) according to the manufacturer's instructions. The purified sample concentration was measured on a Qubit 3 and the concentration was normalized for sequencing. The library was sequenced with Illumina MiniSeq system using an Illumina Mid Output sequencing kit.

### Results

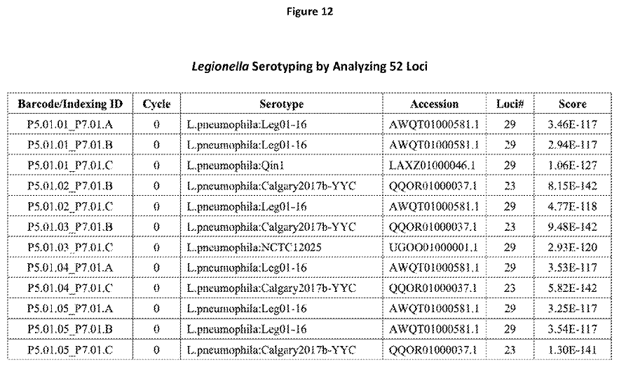

FIG. 12 lists the genotyping results for Legionella samples. The majority of targeted loci were amplified and sequenced. However, the best scored genome matches are about the half loci. Nevertheless, the majority of targeted loci were amplified and sequenced, suggesting that the feasibility of this technology for Legionella genotyping.

### Example 4

### High-Resolution Monkeypox Genotyping and Surveillance NGS Assay

### Materials and Methods

Samples and DNA Extraction: DNA were extracted from clinical samples diagnosed with Monkeypox infection.

Assay Design: The assay was designed to amplify 21 highly polymorphic regions and differentiate monkeypox from poxviruses infecting other mammals. The assay distinguishes monkeypox at different lineages as well as discriminates monkeypox at the same lineage.

Amplification: One-step multiplex barcoding PCR was performed on the extracted DNA by target-specific primers in the presence of barcoded universal primers, DNA polymerase, dNTP and PCR buffer.

Next-generation sequencing: All the amplicons were pooled into one tube. A portion of the samples was then purified with SPRIselect beads (Beckman Coulter, CA, USA) according to the manufacturer's instructions. The purified sample concentration was measured on a Qubit 3 and the concentration was normalized for sequencing. The library was sequenced with Illumina MiniSeq system using an Illumina Mid Output sequencing kit.

### Results

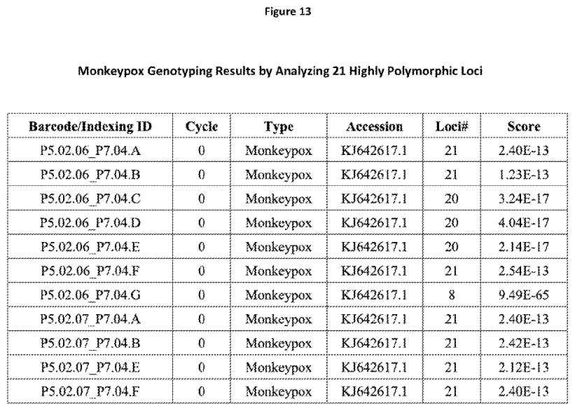

FIG. 13 show shows the genotype results of monkeypox viruses. The table lists the genotype result for monkeypox virus samples, where all best-scored genomes are with accession number KJ642617.1, suggesting that those samples might have similar original source. Sample with barcode/indexing ID P5.02.06_P7.04.G have only 8 loci amplified and sequenced, which is mainly due to low virus titer in the sample.

### Example 5

### High Resolution Listeria Genotyping by Multiplex Barcoding PCR NGS Assay

### Materials and Methods

Samples and DNA Extraction: DNA for eight Listeria samples were extracted from enriched samples.

Assay Design: The Listeria assay was designed to target 69 polymorphic regions across Listeria monocytogenes genome of which 59 target the polymorphic loci, 9 target the traditional MLST loci (abcZ, bgl, cat, dap, dat, ldh, lhk, pgm, sod) and one internal control. The assay also detects other listeria species: L. grayi, L. innocua, L. ivanovii, L. marthii, L. seeligeri, and L. welshimeri.

Amplification: One-step multiplex barcoding PCR was performed on the extracted DNA by target-specific primers in the presence of barcoded universal primers, DNA polymerase, dNTP and PCR buffer.

Next-generation sequencing: All the amplicons were pooled into one tube. A portion of the samples was then purified with SPRIselect beads (Beckman Coulter, CA, USA) according to the manufacturer's instructions. The purified sample concentration was measured on a Qubit 3 and the concentration was normalized for sequencing. The library was sequenced with Illumina MiniSeq system using an Illumina Mid Output sequencing kit.

### Results

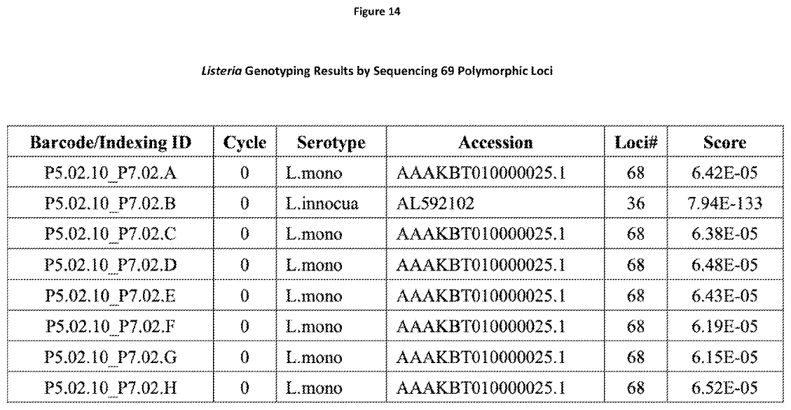

FIG. 14 shows the results of the Listeria Genotyping by barcoding multiplex amplification and variable wide MLST. The table lists the result for listeria samples containing Listeria monocytogenes and Listeria innocua. For Listeria monocytogenes samples, 68 loci can be amplified and sequenced. For Listeria innocua, 36 loci can be amplified and sequenced.

### Example 6

### High Resolution Shiga Toxin-Producing E. Coli (STEC) Genotyping by Multiplex Barcoding PCR NGS Assay

### Materials and Methods

Samples and DNA Extraction: Glycerol stocks of 15 strains were streaked on LB plates, a single colony was picked and grown overnight in LB media, and DNA was extracted using the Qiagen DNeasy kit according to the manufacturer's protocol. E. coli isolates were propagated, and DNA was extracted.

Assay Design: STEC E. coli assay examines 61 polymorphic loci, which include nine loci for housekeeping genes (arcA, aroE, dnaE, espA, gapA, gnd, mdh, ompA, and pgm) used in regular MLST assays. In addition, this assay includes 7 serotype specific (O26, O45, O103, O111, O121, O145 and O157) loci at the wzx gene encoding O-antigen flippase and 11 other loci (uidA, stx1a, stx2a, eaeA, est, elt, aggR, ipaH, bfpA, GFP). Typical enteropathogenic Escherichia coli (EPEC) strains produce bundle-forming pili (BFP). BFP are polymers of bundling, a pilin protein encoded by the bfpA gene found on a large EPEC plasmid.

Amplification: One-step multiplex barcoding PCR was performed on the extracted DNA by target-specific primers in the presence of barcoded universal primers, DNA polymerase, dNTP and PCR buffer.

Next-generation sequencing: All the amplicons were pooled into one tube. A portion of the samples was then purified with SPRIselect beads (Beckman Coulter, CA, USA) according to the manufacturer's instructions. The purified sample concentration was measured on a Qubit 3 and the concentration was normalized for sequencing. The library was sequenced with Illumina MiniSeq system using an Illumina Mid Output sequencing kit.

### Results

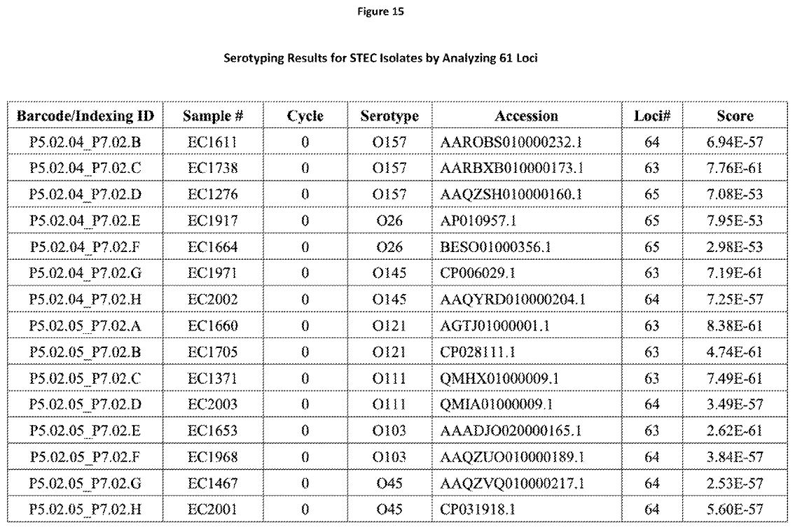

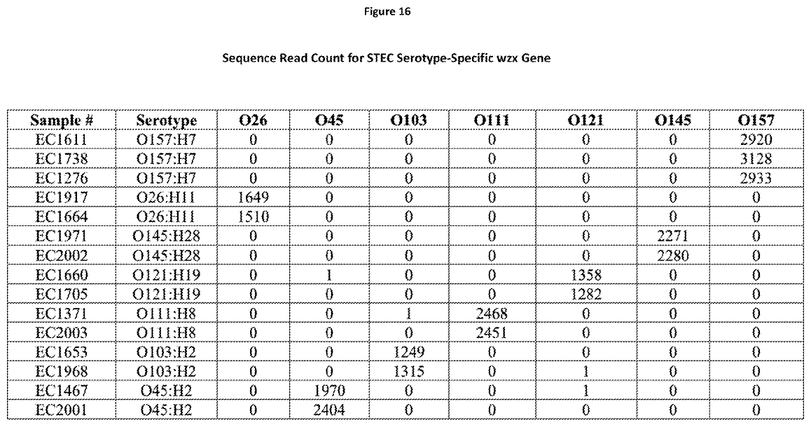

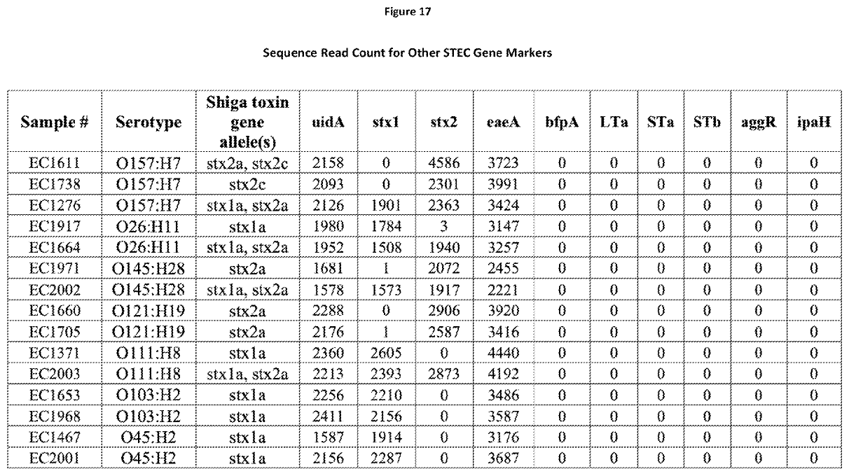

FIG. 15 list the number of loci amplified and sequenced for each sample. Majority of non-serotype-specific loci get amplified and sequenced. O-antigens that are responsible for antigenic specificity of the strain determine the O-serogroup. The wzx (O-antigen flippase) gene was selected as target in this assay for each of E. coli O26, O45, O103, O111, O113, O121, O145, and O157 serogroup. FIG. 16 lists read count of serogroup-specific wzx gene for each sample. The serogroup information for those STEC E. coli isolates is consistent with what the read count predicts. Beside the O-antigens, ten gene markers were selected as targets in this assay to examine virulence gene profiles. FIG. 17 lists the read count of each gene marker for each sample. The read count for stx1 and stx2 information were available for those STEC E. coli isolates, which is consistent with what the read count predicts. In addition, this assay shows that all those STEC E. coli isolates are positive for uidA gene as expected, and positive for eaeA gene as well. This assay shows that all those STEC E. coli isolates are negative for bfpA, LTa, STa, STb, aggR and ipaH genes.

### Example 7

### High Resolution Multiplex Barcoding PCR NGS Assay for Hepatitis A (Hep A) Genotyping

### Materials and Methods

Samples and DNA Extraction: RNA for 3 samples contaminated with HAV was used in this experiment.

Assay Design: The primers for Hepatitis A virus assay were designed to cover 10 conserved regions across Hepatitis A virus genome.

Amplification: Reverse transcription (RT) reaction was first carried out with random RT primers. One-step multiplex barcoding PCR was performed on the cDNA by target-specific primers in the presence of barcoded universal primers, DNA polymerase, dNTP and PCR buffer.

Next-generation sequencing: All the amplicons were pooled into one tube. A portion of the samples was then purified with SPRIselect beads (Beckman Coulter, CA, USA) according to the manufacturer's instructions. The purified sample concentration was measured on a Qubit 3 and the concentration was normalized for sequencing. The library was sequenced with Illumina MiniSeq system using an Illumina Mid Output sequencing kit.

### Results

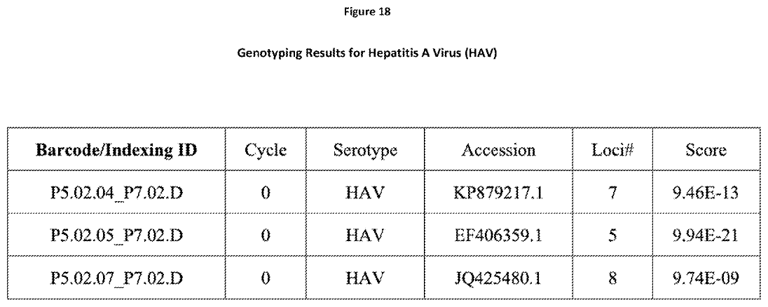

FIG. 18 shows the genotype results of HAV samples. The table lists the result for HAV samples, where between 5 and 8 loci get amplified and sequenced. As HAV is an RNA virus with high mutation rate, the multiple loci sequencing approach increases the chance to detect the HAV virus because even if the assay fails to amplify some loci due to primer mismatch, it can amplify other loci. In addition, the sequence data of multiple loci provides information to track and trace source of contamination by comparing the sequences of those sequenced loci.

### Example 8

### Microbial Identification by Multiplex Barcoding PCR NGS Assay

### Materials and Methods

Samples and DNA Extraction: DNA extracted from environmental samples are used in this experiment.

Assay Design: Primers were designed to target V3 and V4 region of bacterial 16s rRNA. Primers were designed to target fungi internal transcribed region (ITS) between 18s rRNA and 28S rRNA.

Amplification: One-step multiplex barcoding PCR was performed on the extracted DNA by target-specific primers in the presence of barcoded universal primers, DNA polymerase, dNTP and PCR buffer.

Next-generation sequencing: All the amplicons were pooled into one tube. A portion of the samples was then purified with SPRIselect beads (Beckman Coulter, CA, USA) according to the manufacturer's instructions. The purified sample concentration was measured on a Qubit 3 and the concentration was normalized for sequencing. The library was sequenced with Illumina MiniSeq system using an Illumina Mid Output sequencing kit.

### Results

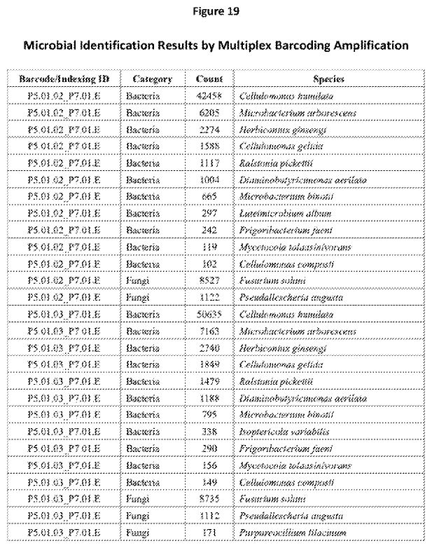

FIG. 19 list the result for environmental samples. Species with more than 100 reads are listed in the table. The assay detected species with a broad range. In addition, the assay detects both bacteria and fungi in the same reaction.

### Example 9

### Single Tube Detection and Genotyping of HIV, HBV and HCV by Multiplex Barcoding PCR NGS Assay

### Materials and Methods

Samples and DNA Extraction: RNA for clinical samples infected with HIV, HCV as well as DNA for clinical sample infected with HBV were used in this experiment.

Assay Design: Primers for 5 amplicons targeting conserved regions of HBV genome, primers for 4 amplicons targeting conserved regions of HCV genome and primers for 5 amplicons targeting conserved regions of HIVI genome, and primers for 4 amplicons targeting conserved regions of HIV2 genome, were designed.

Amplification: RNA was first converted to cDNA with reverse transcription (RT) reaction with random RT primer. One-step multiplex barcoding PCR was performed on cDNA (HIV and HCV) by target-specific primers in the presence of barcoded universal primers, DNA polymerase, dNTP and PCR buffer.

Next-generation sequencing: All the amplicons were pooled into one tube. A portion of the samples was then purified with SPRIselect beads (Beckman Coulter, CA, USA) according to the manufacturer's instructions. The purified sample concentration was measured on a Qubit 3 and the concentration was normalized for sequencing. The library was sequenced with Illumina MiniSeq system using an Illumina Mid Output sequencing kit.

### Results

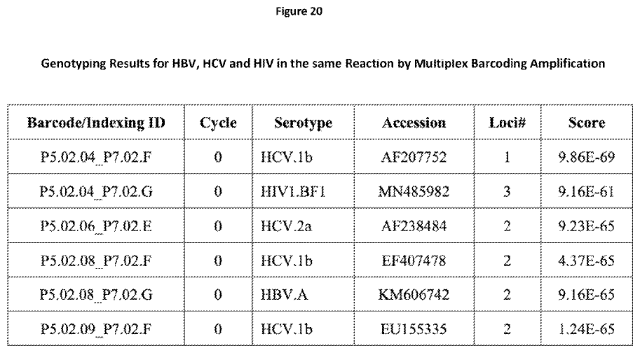

Due to high mutation rate, mismatches between primer and targeting HBV, HCV and HIV genomes are common. To minimize the risk of false negative result, multiloci amplification and sequencing strategy was adopted here for detection and genotyping of HBV, HCV and HIV1/2. FIG. 20 lists the genotyping results, demonstrating the feasibility of this purposed technology for HBV, HCV and HIV1/2 genotyping in a single tube by multiplex barcoding amplification.

### Example 10

### Comprehensive Gene Analysis of BRCA1, BRCA2 and TP53 by Multiplex Barcoding Amplification NGS Assay

### Materials and Methods

Samples and DNA Extraction: 6 human DNA samples were selected for this study of which 1 sample was healthy control and 5 samples had the following mutations respectively: (1) NM_007294.3(BRCA1):c.3700_3704delGTAAA, 2) (p.Val1234Glnfs); (2) M_000059.4(BRCA2):c.6468_6469del (p.Gln2157fs); (3) NM_007294.3(BRCA1):c.5266dupC (p.Gln1756Profs); (4) NM_007294.3(BRCA1):c.302-2A>C, and 5) NM_000059.3(BRCA2):c.9097dupA (p.Thr3033 Asnfs). As reference control, BRCA Somatic Multiplex I (gDNA) control from Horizon Discovery (Waterbeach, UK) was used, which includes 15 variants with allelic frequencies between 7.5% to 60%.

Assay Design: primers were designed to cover all coding region of both BRCA1, BRCA2 and TP53 gene. Overlapping amplicons were designed to cover exons larger than 300 bp. Two PCR reactions were carried out for each sample to separate overlapping amplicons.

Amplification: One-step multiplex barcoding PCR was performed on the extracted DNA by target-specific primers in the presence of barcoded universal primers, DNA polymerase, dNTP and PCR buffer.

Next-generation sequencing: All the amplicons were pooled into one tube. A portion of the samples was then purified with SPRIselect beads (Beckman Coulter, CA, USA) according to the manufacturer's instructions. The purified sample concentration was measured on a Qubit 3 and the concentration was normalized for sequencing. The library was sequenced with Illumina MiniSeq system using an Illumina Mid Output sequencing kit.

### Results

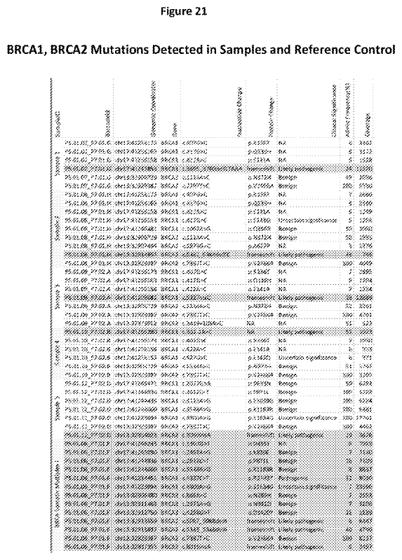

FIG. 19A depicts screenshot of alignments of reads onto BRCA1 exons presented by integrative genomics viewer (IGV, https://software.broadinstitute.org/software/igv/) and FIG. 21 shows the mutations detected in the test samples. The results show that all known mutations for each sample as well as BRCA Somatic Multiplex I (gDNA) control reference sample from Horizon Discovery were detected in the experiment, indicating that the assay is both sensitive and specific to detect somatic mutations in clinical samples.

### Example 11

### STR Sequencing by Multiplex Barcoding Amplification NGS Assay

### Materials and Methods

Genomic DNA from two human samples were used in this assay. An additional replicate was performed for sample 2 to test the reproducibility of the assay.

Assay Design: PCR primers were designed to surround the D1S1656, D10S1248, D10S1435, D10S2325, D13S317, D13S325, D15S659, D16S539, D17S1301, D18S1364, D18S51, D19S433, D20S482, D21S11 STR loci.

Amplification: One-step multiplex barcoding PCR was performed on the extracted DNA by target-specific primers in the presence of barcoded universal primers, DNA polymerase, dNTP and PCR buffer.

Next-generation sequencing: All the amplicons were pooled into one tube. A portion of the samples was then purified with SPRIselect beads (Beckman Coulter, CA, USA) according to the manufacturer's instructions. The purified sample concentration was measured on a Qubit 3 and the concentration was normalized for sequencing. The library was sequenced with Illumina MiniSeq system using an Illumina Mid Output sequencing kit.

### Results

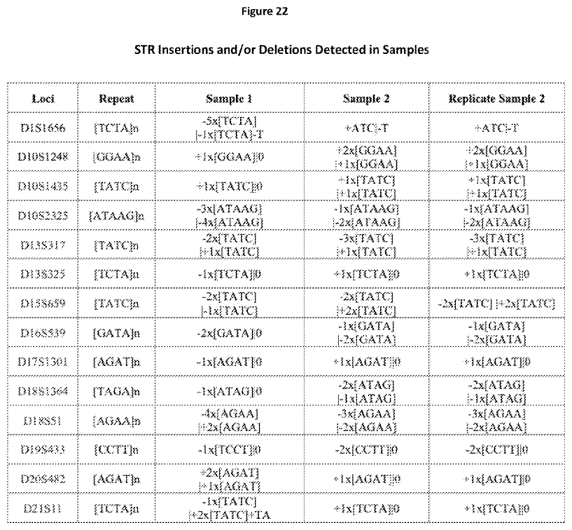

FIG. 22 lists the insertion and/or deletion detected in the two samples. Note that (−) symbol indicates deletion and (+) symbol indicates insertion. The number after either (+) or (−) indicates the number of copies of repeat unit detected in the assay. Two patterns are shown for each sample. All those insertions and/or deletions are related to hg19 genome reference.

The insertion and/or deletion pattern between two replicates samples are consistent with each other. Most of the insertion and/or deletion patterns match the STR pattern for the loci. However, there are a few exceptions such as deletion of T in D1S1656 locus, and insertion of TA at D21S11 locus in Sample 1. The assay shows the feasibility of purposed technology can be used to amplify STR loci for forensic testing.

Now, therefore, the following is claimed:

1. A method of screening and analyzing at least one sample for food, animal, human and environmental pathogens, comprising the steps of:
for each sample, hybridizing a plurality of target-specific primers with nucleic acid from the sample in the presence of barcoded universal primers to form a test reaction in a single reaction container, wherein at least one target-specific primer is configured to bind to at least one target nucleic acid sequence;
subjecting each test reaction to amplification conditions to generate amplicons;
pooling amplicons from each sample;
subjecting at least a portion of the pooled amplicons to bead cleanup to form enriched amplicons; and
sequencing the pooled and enriched amplicons, formed from each sample, by next-generation sequencing.

2. The method of claim 1, wherein each barcoded universal primer comprises:
a universal priming portion at the 3′-end;
a barcode portion in the middle; and
a universal priming portion at the 5′-end.

3. The method of claim 1, wherein each target-specific primer comprises a specific sequence portion directed to a target nucleic acid sequence and a universal priming portion.

4. The method of claim 1, wherein each sample is obtained from a subject, food, one or more plants, or an environmental source.

5. The method of claim 1, wherein the target-specific primers comprise primers configured to amplify at least 20 variable regions of at least a bacterium, fungi, parasite or virus for identification, genotyping, subtyping and detection of co-presence of multiple isolates.

6. The method of claim 1, further comprising mapping-and-counting for microbial and viral typing, subtyping, and surveillance of multiple pathogen genomes, comprising the additional steps of: (1) determining the score for a locus A for a first genome as the ratio between the number of unique reads mapped onto the first genome's locus A and the total number of unique reads mapped onto the locus A for the first genome and at least one other genome wherein if no read is mapped to the first genome's locus A, presetting the score to a de minimis number; repeating the determination step of part (1) for at least one other locus; and (3) determining an overall score for the first genome by multiplying the scores for all tested loci from steps (1) and (2).

7. The method of claim 6, further comprising the steps of: (a) determining the genomes with the highest overall score on any remaining reads; (b) ending the assessment if the number of non-empty loci for highest-scored genome is less than a preset cutoff, (c) outputting the highest-scored genomes; (d) removing reads mapped to all then-highest scored genomes; and (e) repeating steps (a)-(d) until the assessment ends in accordance with step (b).

8. The method of claim 7, wherein the preset cutoff is at least 10.

9. The method of claim 1, where in the target-specific primers comprise primers configured to amplify multiple variable regions of at least a bacteria, fungi, parasite or virus for genotyping, subtyping, detection and identification of multiple genotypes or subtypes of the same species or different species in the same sample.

10. The method of claim 1, wherein the target-specific primers comprise primers configured to amplify and detect target sequences of at least one species/type/subtype of bacteria, fungi, parasites, or viruses in the same sample.

11. The method of claim 1, wherein the sample comprises at least one microbial and viral species, strain/type, or sub-strain/subtype, which are differentiated.

12. The method of claim 1, wherein the target-specific primers comprise primers configured to amplify and analyze target sequences related to forensic testing.

13. The method of claim 1, further comprising the step of pooling the enriched amplicons from each sample prior to sequencing.

14. The method of claim 1, further comprising the step of quantifying each type and species in each sample after sequencing the enriched amplicons.

15. The method of claim 1, wherein the test reaction comprises a polymorphic gene with unique sequence for an internal control.

16. A method of screening at least one sample for food, human, animal and environmental pathogens, comprising the steps of:
for each sample, hybridizing a plurality of target-specific primers with nucleic acid from the sample in the presence of barcoded universal primers to form a test reaction in a single reaction container, wherein at least one target-specific primer is configured to bind to at least one target sequence selected from the group consisting of: bacteria, fungi, parasites or virus, wherein each barcoded universal primer comprises:
a universal priming portion at the 3′-end;
a barcode portion in the middle; and
a universal priming portion at the 5′-end; and wherein each target-specific primer comprises a specific sequence portion directed to a target nucleic acid sequence and a universal priming portion;
subjecting each test reaction to amplification conditions to generate amplicons;
subjecting amplicons from each sample to pool;
subjecting at least a portion of the pooled amplicons generated from each sample to bead cleanup to form pooled and enriched amplicons; and
sequencing the pooled and enriched amplicons, formed from each sample, by next-generation sequencing.

17. A method of screening at least one sample for forensic DNA analysis, cancer, or genetic disorders, comprising the steps of:
for each sample, hybridizing a plurality of target-specific primers with nucleic acid from the sample in the presence of barcoded universal primers to form a test reaction in a single reaction container, wherein at least one target-specific primer is configured to bind to at least one target sequence, wherein each barcoded universal primer comprises:
a universal priming portion at the 3′-end;
a barcode portion in the middle; and
a universal priming portion at the 5′-end; and wherein each target-specific primer comprises a specific sequence portion directed to a target nucleic acid sequence and a universal priming portion;
subjecting each test reaction to amplification conditions to generate amplicons;
subjecting amplicons from each sample to pool;
subjecting at least a portion of the pooled amplicons generated from each sample to bead cleanup to form enriched amplicons; and
sequencing the enriched amplicons, formed from each sample, by next-generation sequencing.

18. A kit, comprising multiplex target-specific primers configured to bind to target sequences specific to: biological samples related to cancer, genetic disorders, forensic testing, and microbial and viral species.
